(19) United States
(12) Patent Application Publication
(10) Pub. No.: US 2004/0168208 A2
(43) Pub. Date: Aug. 26, 2004

(54) PRODUCTION OF BUTYRYLCHOLINESTERASES IN TRANSGENIC ANIMALS

(75) Inventors: Costas Karatzas, Quebec, (CA); Yue-Jin Huang, Quebec, (CA); Anthoula Lazaris, Quebec, (CA)

Correspondence Address: DARBY & DARBY P.C., P. O. BOX 5257, NEW YORK, NY, 10150-5257, US

(73) Assignee: Nexia Biotechnologies, Inc., Quebec (CA)

(21) Appl. No.: 10/326,892

(22) Filed: Dec. 20, 2002

Related U.S. Application Data
(60) Provisional application No. 60/344,295, filed on Dec. 21, 2001.

(51) Int. Cl. A01K 67/027

(52) U.S. Cl. 800/7; 800/14; 800/18

(57) ABSTRACT

### Abstract of the Disclosure

The present invention provides methods for the large-scale production of recombinant butyrylcholinesterase in cell culture, and in the milk and/or urine of transgenic mammals. The recombinant butyrylcholinesterases of this invention can be used to treat and/or prevent organophosphate pesticide poisoning, nerve gas poisoning, cocaine intoxication, and succinylcholine-induced apnea.

### Detailed Description of the Invention

### CROSS REFERENCE TO RELATED APPLICATIONS

This application claims priority to provisional U.S. application number 60/344,295 filed December 21, 2001 under 35 U.S.C. § 119(e), which is incorporated by reference.

### FIELD OF THE INVENTION

The present invention provides methods for the large-scale production of recombinant butyrylcholinesterase in cell culture, and in the milk and/or urine of transgenic mammals. The recombinant butyrylcholinesterases of this invention can be used to treat and/or prevent organophosphate pesticide poisoning, nerve gas poisoning, cocaine intoxication, and succinylcholine-induced apnea.

### BACKGROUND OF THE INVENTION

The general term cholinesterase (ChE) refers to a family of enzymes involved in nerve impulse transmission. The major function of ChE enzymes is to catalyze the hydrolysis of the chemical compound acetylcholine at the cholinergic synapses. Electrical switching centers, called synapses, are found throughout the nervous systems of humans, other vertebrates and insects. Muscles, glands, and neurons are stimulated or inhibited by the constant firing of signals across these synapses. Stimulating signals are carried by the neurotransmitter acetylcholine, and discontinued by the action of ChE enzymes, which cause hydrolytic breakdown of acetylcholine. These chemical reactions are going on all the time at a very fast rate, with acetylcholine causing stimulation and ChE enzymes ending the signals. The action of ChE allows the muscle, gland, or nerve to return to its resting state, ready to receive another nerve impulse if need be.

If cholinesterase-inhibiting substances such as organophosphate compounds or carbamate insecticides or drugs are present, this system is thrown out of balance. These cholinesterase-inhibiting substances prevent the breakdown of acetylcholine, resulting in a buildup of acetylcholine, thereby causing hyperactivity of the nervous system. Acetylcholine is not destroyed and continues to stimulate the muscarinic receptor sites (exocrine glands and smooth muscles) and the nicotinic receptor sites (skeletal muscles). Exposure to cholinesterase-inhibiting substances can cause symptoms ranging from mild (twitching, trembling) to severe (paralyzed breathing, convulsions), and in extreme cases, death, depending on the type and amount of cholinesterase-inhibiting substances involved. The action of cholinesterase-inhibiting substances such as organophosphates and carbamates makes them very effective as pesticides for controlling insects and other pests. Unfortunately, when humans breathe or are otherwise exposed to these compounds, they are subjected to the same negative effects. Indeed, the devastating impact of certain cholinesterase-inhibiting substances on humans has led to the development of these compounds as "nerve gases" or chemical warfare agents.

Cholinesterases are classified into two broad groups, depending on their substrate preference and sensitivity to selective inhibitors. Those enzymes which preferentially hydrolyze acetyl esters such as acetylcholine, and whose enzymatic activity is sensitive to the chemical inhibitor BW 284C51, are called acetylcholinesterases (AChE), or acetylcholine acetylhydrolase, (EC 3.1.1.7). Those enzymes which preferentially hydrolyze other types of esters such as butyrylcholine, and whose enzymatic acticity is sensitive to the chemical inhibitor tetraisopropylpyrophosphoramide (also known as iso-OMPA), are called butyrylcholinesterases (BChE, EC 3.1.1.8). BChE is also known as pseudocholinesterase or non-specific cholinesterase. Further classifications of ChE's are based on charge, hydrophobicity, interaction with membrane or extracellular structures, and subunit composition.

Acetylcholinesterase (AChE), also known as true, specific, genuine, erythrocyte, red cell, or Type I ChE, is a membrane-bound glycoprotein and exists in several molecular forms. It is found in erythrocytes, nerve endings, lungs, spleen, and the gray matter of the brain. Butyrylcholinesterase (BChE), also known as plasma, serum, benzoyl, false, or Type II ChE, has more than eleven isoenzyme variants and preferentially uses butyrylcholine and benzoylcholine as in vitro substrates. BChE is found in mammalian blood plasma, liver, pancreas, intestinal mucosa, the white matter of the central nervous system, smooth muscle, and heart. BChE is sometimes referred to as serum cholinesterase as opposed to red cell cholinesterase (AChE).

AChE and BChE exist in parallel arrays of multiple molecular forms composed of different numbers of catalytic and non-catalytic subunits. Both enzymes are composed of subunits of about 600 amino acids each, and both are glycosylated. AChE may be distinguished from the closely related BChE by its high specificity for the acetylcholine substrate and sensitivity to selective inhibitors. While AChE is primarily used in the body to hydrolyze acetylcholine, the specific function of BChE is not as clear. BChE has no known specific natural substrate, although it also hydrolyzes acetylcholine.

Despite the devastating effects of certain cholinesterase-inhibiting substances on humans, these compounds are not without therapeutic uses. Cholinesterase-inhibiting drugs are employed to treat a wide variety of diseases including Alzheimer's and Parkinson's diseases, glaucoma, multiple sclerosis, and myasthenia gravis. The cholinesterase-inhibiting compound succinyl choline is commonly used as a short-acting muscle relaxant in surgical operations. In particular, it is used during tracheal intubation in the administration of inhalation anesthetics.

Certain human individuals have a mutant BChE gene which lacks the ability to hydrolyze succinyl choline. In rare individuals the complete BChE gene is missing. Neither of these gene defects results in gross physiological consequence. However, these individuals suffer from prolonged apnea following administration of succinyl choline. Unfortunately, there are no rapid, simple, and routine methods to detect and characterize the atypic forms of the enzyme prior to surgery.

Poisoning with organophosphate agents is a severe problem facing military personnel who may encounter lethal doses of these compounds in chemical warfare situations. The use of organophosphate compounds in war and as pesticides has resulted over the past 40 years in a rising number of cases of acute and delayed intoxication, resulting in damage to the peripheral and central nervous systems, myopathy, psychosis, general paralysis, and death. It is estimated that 19,000 deaths occur out of the 500,000 to 1 million annual pesticide-related poisonings. In addition to these overt symptoms, animal studies have shown that administration of the organophosphate methyl parathion suppressed growth and induced ossification in both mice and rats. In humans, malformations of the extremities and fetal death were correlated with exposure to methyl parathion in 18 cases. In addition, a neonatal lethal syndrome of multiple malformations was reported in women exposed to unspecified pesticides early in pregnancy.

Nerve agents are the most toxic chemical warfare agents. These compounds are related to organophosphorus insecticides, in that they are both esters of phosphoric acid. The major nerve agents are GA (tabun), GB (sarin), GD (soman), GF, and VX. VX is a persistent substance which can remain on material, equipment, and terrain for long periods. Under temperate conditions, nerve agents are clear colorless liquids.

Nerve agents exert their biological activity by inhibiting the cholinesterase enzymes. In cases of moderate to severe organophospate poisoning, the levels of both AChE and BChE activity are reduced. Mild poisoning occurs when cholinesterase activity is 20-50% of normal; moderate poisoning occurs when activity is 10-20% of normal; severe poisoning is characterized by activity of less than 10% of normal. Severe neuromuscular effects are observed when ChE activity levels drop below 20% of normal, while levels near zero are generally fatal.

Present treatment of organophosphate poisoning consists of post-exposure intravenous or intramuscular administration of various combinations of drugs, including carbamates ( e.g ., pyridostigmine), anti-muscarinics ( e.g., atropine), and ChE-reactivators such pralidoxime chloride (2-PAM, Protopam). A diazopan compound may also be administered. Although this drug regimen is effective in preventing death from organophosphate poisoning, it is not effective in preventing convulsions, performance deficits, or permanent brain damage. In addition, a post-exposure drug regimen is often useless because even a small dose of an organophosphate chemical warfare agent can cause instant death. These drawbacks have led to the investigation of cholinesterase enzymes for the treatment of organophosphate exposure. Post-exposure symptoms can be prevented by pretreatment with cholinesterases, which act to sequester the toxic organophosphates before they reach their physiological targets.

The use of cholinesterases as pre-treatment drugs has been successfully demonstrated in animals, including non-human primates. For example, pretreatment of rhesus monkeys with fetal bovine serum-derived AChE or horse serum-derived BChE protected them against a challenge of two to five times the LD ₅₀ of pinacolyl methylphosphonofluoridate (soman), a highly toxic organophosphate compound used as a war-gas (Broomfield et al., J. Pharmacol. Exp. Ther. (1991) 259:633-638; Wolfe et al. , Toxicol. Appl. Pharmacol. (1992) 117(2):189-193). In addition to preventing lethality, the pretreatment prevented behavioral incapacitation after the soman challenge, as measured by the serial probe recognition task or the equilibrium platform performance task. Administration of sufficient exogenous human BChE can protect mice, rats, and monkeys from multiple lethal-dose organophosphate intoxication [see, for example, Raveh et al., Biochemical Pharmacology (1993) 42:2465-2474; Raveh et al., Toxicol. Appl. Pharmacol. (1997) 145:43-53; Allon et al., Toxicol. Sci. (1998) 43:121-128]. Purified human BChE has been used to treat organophosphate poisoning in humans, with no significant adverse immunological or psychological effects (Cascio et al., Minerva Anestesiol (1998) 54:337).

Titration of organophosphates both in vitro and in vivo demonstrates a 1:1 stoichiometry between organophosphate-inhibited enzymes and the cumulative dose of the toxic nerve agent. The inhibition of ChE by a organophosphate agent is due to the formation of a stable stoichiometric (1:1) covalent conjugate of the organophosphate with the ChE active site serine. Covalent conjugation is followed by a parallel competing reaction, termed "aging", wherein the inhibited ChE is transformed into a form that cannot be regenerated by the commonly used reactivators. These reactivators, such as active-site directed nucleophiles ( e.g., quaternary oximes), normally detach the phosphoryl moiety from the hydroxyl group of the active site serine. The aging process is believed to involve dealkylation of the covalently bound organophosphate group, and renders therapy of intoxication by certain organophosphates such as sarin, soman, and DFP exceedingly difficult.

Despite the promise of cholinesterases as drugs to protect against organophosphate poisoning, their widespread use is not currently possible due to the limited supply of these enzymes. Because of the 1:1 stoichiometry required to provide protection, large quantities of cholinesterase enzymes are needed for effective treatment. The only practical source of these enzymes at present is by extraction from human plasma (see, e.g., U.S. Patent No. 5,272,080 to Lynch). It is estimated that the number of doses needed for military purposes alone far exceeds the available supplies. In addition, there is a huge demand in the pesticide and agricultural industries for effective pre- and post-treatment of humans subject to organophosphate and carbamate pesticide exposure. The stockpiling of organophosphate chemical warfare agents has led to a need to find ways to detoxify such stocks, including decontamination of land where these chemicals have been stored. In addition, military equipment used in environments where chemical warfare agents have been released must be decontaminated to remove the chemical warfare agent before the equipment can be used again.

In addition to its efficacy in hydrolyzing organophosphate toxins, there is strong evidence that BChE is the major detoxicating enzyme of cocaine (Xie, et al. , Molec. Pharmacol. (1999) 55:83-91). Cocaine abuse is a major medical problem in the United States. It is estimated that there are approximately 5 million habitual users of cocaine. The number of cocaine-related emergency room visits is about 100,000 annually. Life-threatening symptoms due to cocaine intoxication include grand-mal seizures, cardiac arrest, stroke, and drug-induced psychosis. Individual response to cocaine is highly variable, with death reported after exposure to as little as 20mg and survival reported with daily use of as much as 10g. Cocaine is metabolized by three major routes: hydrolysis by BChE to form ecgonine methyl ester, N-demethylation from norcocaine, and nonenzymatic hydrolysis to form benzoylcholine. Studies have shown a direct correlation between low BChE levels and episodes of life-threatening cocaine toxicity. A recent study has confirmed that a decrease of cocaine half-life in vitro correlated with the addition of purified human BChE.

In view of the significant pharmaceutical potential of ChE enzymes, research has focused on development of recombinant methods to produce them. Recombinant enzymes, as opposed to those derived from plasma, have a much lower risk of transmission of infectious agents, including viruses such as hepatitis C and HIV.

The cDNA sequences have been cloned for both human AChE (see U.S. Patent No. 5,595,903) and human BChE (see U.S. Patent No. 5,215,909 to Soreq; Prody, et al. Proc. Natl. Acad. Sci. USA (1987) 84:3555-3559; McTiernan, et al. Proc. Natl. Acad. Sci. USA (1987) 84:6682-6686). In addition, a number of variants of AChE and BChE have been reported. For example, U.S. Patent No. 5,248,604 to Fischer discloses a non-glycosylated variant of human AChE. Various forms of human AChE resulting from alternate splicing, as well as transgenic frogs and mice that express AChE enzymes, are disclosed in U.S. Patent Nos. 5,932,780 and 6,025,183 to Soreq. These transgenic AChE animals are reported to have utility as assay systems for testing efficacy of anti-cholinesterase drugs, and the toxicity of anti-cholinesterase poisons, including organophosphorous compounds. The amino acid sequence of wildtype human BChE, as well as of several BChE variants with single amino acid changes, is set forth in U.S. Patent No. 6,001,625 to Broomfield et al.

Recombinant expression of BChE has been reported in E. coli (Masson, P., "Expression and Refolding of Functional Human BChE from E. coli ," Multiple Approaches to Cholinesterase Functions (Eds. Shafferaman, A. and Velan, B.), Plenum, New York (1990) pp. 49-52); microinjected Xenopus laevis oocytes (U.S. Patent No. 5,215,909 to Soreq; Soreq et al. , J. Biol. Chem. (1989) 264:10608-10613; Soreq et al. , EMBO Journal (1984) 3(6):1371-1375); insect cell lines in vitro and larvae in vivo (Platteborze and Broomfield, Biotechnol Appl Biochem (2000) 31:225-229); the silkworm Borbyx mori (Wei, et al. Biochem Pharmacol (2000) 60(1):121-126); and in mammalian COS cells (Platteborze and Broomfield Biotechnol Appl Biochem (2000) 31:225-229) and CHO cells (Masson, et al. J Biol Chem (1993) 268(19):14329-41; Lockridge, et al. Biochemistry (1997) 36(4):786-795; Blong, et al. Biochem. J (1997) 327:747-757; and Altamirano, et al. J Neurochemistry (2000) 74:869-877). However, many of these reported recombinantly produced BChE preparations have thus far showed little or no in vivo enzyme activity.

Notably, none of the recombinant expression systems reported to date have the ability to produce BChE in quantities sufficient to allow development of the enzyme as a drug to treat such conditions as organophosphate poisoning, post-surgical apnea, or cocaine intoxication. Thus, there is a need in the art for a recombinant system capable of expressing large quantities of BChE that demonstrate significant in vivo enzymatic activity, so that the huge pharmaceutical potential of these enzymes can be realized.

### SUMMARY OF THE INVENTION

The present inventors have discovered methods for producing large quantities of recombinant butyrylcholinesterase in the milk of lactating transgenic mammals, and in the urine of transgenic mammals. The methods of the invention for the first time allow sufficient quantities of the BChE enzyme to be produced so as to permit practical development of this enzyme for prevention and/or treatment for organophosphorus poisoning, cocaine intoxication, and succinyl choline-induced apnea.

The present invention is directed to non-human transgenic mammals that upon lactation, express a BChE enzyme in their milk, where the genomes of the mammals comprise a DNA sequence encoding a BChE enzyme, operably linked to a mammary gland-specific promoter, and a signal sequence that provides secretion of the BChE enzyme into the milk of the mammal. In preferred embodiments, the mammary gland-specific promoter is a casein promoter or a whey acidic protein (WAP) promoter. In preferred embodiments, the transgenic mammals are goats or rodents.

The present invention is also directed to non-human transgenic mammals that express a BChE enzyme in their urine, where the genomes of the mammals comprise a DNA sequence encoding a BChE enzyme, operably linked to a urinary endothelium-specific promoter, and a signal sequence that provides secretion of the BChE enzyme into the urine of the mammal. In preferred embodiments, the urinary endothelium-specific promoter is a uroplakin promoter or a uromodulin promoter. In preferred embodiments, the transgenic mammals are goats or rodents.

In further embodiments, the invention is directed to such transgenic mammals, where the genomes of the mammals further comprise a DNA sequence encoding a glycosyltransferase, operably linked to a mammary gland-specific or a urinary endothelium-specific promoter, and a signal sequence that provides secretion of the glycosyltransferase. The BChE enzyme and the glycosyltransferase may be encoded together in a single, bi-cistronic expression construct. Alternatively, the BChE enzyme and the glycosyltransferase are encoded in separate expression constructs, which are both introduced into the genome of the mammal.

In another aspect the present invention is directed to a genetically-engineered DNA sequence, which comprises: (i) a sequence encoding a BChE enzyme; (ii) a mammary gland-specific promoter that directs expression of the BChE enzyme; and (iii) at least one signal sequence that provides secretion of the expressed BChE enzyme. In preferred embodiments, the mammary gland-specific promoter is a WAP (whey acidic protein) promoter or a casein promoter. The invention also contemplates a non-human mammalian embryo or mammalian cell that comprises such a DNA sequence, especially where the cell is a MAC-T (mammary epithelial) cell, embryonic stem cell, embryonal carcinoma cell, primordial germ cell, oocyte, or sperm. The present invention is also directed to a method for making such a genetically-engineered DNA sequence, which method comprises joining a sequence encoding a BChE enzyme with a mammary gland-specific promoter the directs expression of the BChE enzyme and at least one signal sequence that provides secretion of the expressed BChE enzyme.

In another aspect the present invention is directed to a genetically-engineered DNA sequence, which comprises: (i) a sequence encoding a BChE enzyme; (ii) a urinary endothelium-specific promoter that directs expression of the BChE enzyme; and (iii) at least one signal sequence that provides secretion of the expressed BChE enzyme. In preferred embodiments, the urinary endothelium-specific promoter is a uroplakin promoter or a uromodulin promoter. The invention also contemplates a non-human mammalian embryo or mammalian cell that comprises such a DNA sequence, especially where the cell is a BHK (baby hamster kidney) cell, embryonic stem cell, embryonal carcinoma cell, primordial germ cell, oocyte, or sperm. The present invention is also directed to a method for making such a genetically-engineered DNA sequence, which method comprises joining a sequence encoding a BChE enzyme with a urinary endothelium-specific promoter the directs expression of the BChE enzyme and at least one signal sequence that provides secretion of the expressed BChE enzyme.

The invention is also directed to a method for producing a transgenic mammal that upon lactation secretes a BChE enzyme in its milk, which method comprises allowing an embryo, into which at least one genetically-engineered DNA sequence, comprising (i) a sequence encoding a BChE enzyme; (ii) a mammary gland-specific promoter; and (iii) at least one signal sequence that provides secretion of the BChE enzyme into the milk of the mammal, has been introduced, to grow when transferred into a recipient female mammal, resulting in the recipient female mammal giving birth to the transgenic mammal. In one embodiment, this method further comprises introducing the genetically-engineered DNA sequence into a cell of the embryo, or into a cell that will form at least part of the embryo. In specific embodiments, introducing the genetically-engineered DNA sequence comprises pronuclear or cytoplasmic microinjection of the DNA sequence; combining a mammalian cell stably transfected with the DNA sequence with a non-transgenic mammalian embryo; or introducing the DNA sequence into a non-human mammalian oocyte; and activating the oocyte to develop into an embryo.

The invention is further directed to a method for producing a transgenic mammal that upon lactation secretes a BChE enzyme in its milk, which method comprises cloning or breeding of a transgenic mammal, the genome of which comprises a DNA sequence encoding a BChE enzyme, operably linked to a mammary gland-specific promoter, wherein the sequence further comprises a signal sequence that provides secretion of the BChE enzyme into the milk of the mammal.

The invention is also directed to a method for producing a transgenic mammal that secretes a BChE enzyme in its urine, which method comprises allowing an embryo, into which at least one genetically-engineered DNA sequence, comprising (i) a sequence encoding a BChE enzyme; (ii) a urinary endothelium-specific promoter; and (iii) at least one signal sequence that provides secretion of the BChE enzyme into the urine of the mammal, has been introduced, to grow when transferred into a recipient female mammal, resulting in the recipient female mammal giving birth to the transgenic mammal. In one embodiment, this method further comprises introducing the genetically-engineered DNA sequence into a cell of the embryo, or into a cell that will form at least part of the embryo. In specific embodiments, introducing the genetically-engineered DNA sequence comprises pronuclear or cytoplasmic microinjection of the DNA sequence; combining a mammalian cell stably transfected with the DNA sequence with a non-transgenic mammalian embryo; or introducing the DNA sequence into a non-human mammalian oocyte; and activating the oocyte to develop into an embryo.

The invention is further directed to a method for producing a transgenic mammal that secretes a BChE enzyme in its urine, which method comprises cloning or breeding of a transgenic mammal, the genome of which comprises a DNA sequence encoding a BChE enzyme, operably linked to a urinary endothelium-specific promoter, wherein the sequence further comprises a signal sequence that provides secretion of the BChE enzyme into the urine of the mammal.

The invention is directed to a method for producing a BChE enzyme, which method comprises: (a) inducing or maintaining lactation of a transgenic mammal, the genome of which comprises a DNA sequence encoding a BChE enzyme, operably linked to a mammary gland-specific promoter, where the sequence further comprises a signal sequence that provides secretion of the BChE enzyme into the milk of the mammal; and (b) extracting milk from the lactating mammal. In a specific embodiments, this method may comprise the additional steps of isolating the BChE enzyme, or isolating and purifying the BChE enzyme.

Accordingly, the invention is also directed to the milk of a non-human mammal comprising a human BChE enzyme, and to milk comprising a BChE enzyme produced by a transgenic mammal according to the methods of the invention.

The invention is also directed to a method for producing a BChE enzyme, which method comprises, extracting urine from a transgenic mammal, the genome of which comprises a DNA sequence encoding a BChE enzyme, operably linked to a urinary endothelium-specific promoter, where the sequence further comprises a signal sequence that provides secretion of the BChE enzyme into the urine of the mammal. In specific embodiments, this method may comprise the additional steps of isolating the BChE enzyme, or isolating and purifying the BChE enzyme.

Accordingly, the invention is also directed to the urine of a non-human mammal comprising a human BChE enzyme, and to urine comprising a BChE enzyme produced by a transgenic mammal according to the methods of the invention.

The invention is also direct to a method for producing a BChE enzyme in a culture of MAC-T or BHK cells, which method comprises: (a) culturing said cells, into which a DNA sequence comprising (i) a DNA sequence encoding a BChE enzyme, (ii) a promoter that provides expression of the encoded BChE enzyme within said cells, and (iii) a signal sequence that provides secretion of the BChE enzyme into the cell culture medium, has been introduced; (b) culturing the cells; and (c) collecting the cell culture medium of the cell culture. In specific embodiments, this method may comprises the additional steps of isolating the BChE enzyme, or isolating and purifying the BChE enzyme. In a preferred embodiment of this method, the cells are MAC-T cells and at least 50% of the produced BChE enzyme is in tetramer form. Accordingly, the invention also encompasses cell culture medium comprising a BChE enzyme produced by cultured MAC-T or BHK-1 cells according to this method.

The invention also encompasses cell culture medium from a culture of mammalian cells, which medium comprises a BChE enzyme, wherein at least 50% of the BChE enzyme is in tetramer form.

The invention also provides a method for producing a pharmaceutical composition, which comprises combining a BChE enzyme produced by a transgenic mammal or cultured MAC-T or BHK cells with a pharmaceutically acceptable carrier or excipient. Accordingly, the invention is further directed to methods for the treatment of organophosphate poisoning, post-surgical succinyl choline-induced apnea, and cocaine intoxication, which methods comprise administering to a subject in need thereof a therapeutically effective amount of a pharmaceutical composition produced by the methods of the invention.

The invention also encompasses a transgenic non-human mammal capable of expressing BChE enzyme in both its milk and its urine. The genome of said transgenic mammal comprises (a) a DNA sequence encoding a BChE enzyme, operably linked to a mammary gland-specific promoter, and further comprising a signal sequence that provides secretion of the BChE enzyme into the milk of the mammal; and (b) a DNA sequence encoding a BChE enzyme, operably linked to a urinary endothelium-specific promoter, and further comprising a signal sequence that provides secretion of the BChE enzyme into the urine of the mammal. These two DNA sequences may be encoded in a single, bi-cistronic expression construct, or in independent expression constructs.

### BRIEF DESCRIPTION OF THE DRAWINGS

FIGURES 1A and 1B depict the cDNA and translated amino acid sequence of wild-type human BChE. The signal sequence is in bold. The signal peptide, which is cleaved during processing to produce the mature BChE protein, is underlined. Amino acids are represented by the standard one-letter code. * indicates the STOP codon.

FIGURE 2 depicts the locations of altered residues in some naturally occuring human BChE variants (See also Table 1). Amino acids are represented by the standard one-letter code. One letter codes shown above the amino acid sequence represent the type of variant as follows: A = atypical; F = fluoride resistant; H , J , and K = H, J, and K variants; N = unstable variant; and S = Silent (no or very low activity) variants. Asterisks ( * ) shown below the amino acid sequence mark the residues of the catalytic triad.

FIGURE 3 depicts a non-reducing BChE-activity gel of condition serum-free cell culture media from stably transfected cell lines expressing recombinant BChE. Conditioned, serum free media was from: Lane 1) MAC-T cells, untransfected control; Lane 2) MAC-T cells stably transfected with pCMV/IgKBChE; Lane 3) MAC-T cells stably transfected with pCMV/BChE/hSA; Lane 4) BHK cells, untransfected control; Lane 5) BHK cells stably transfected with pCMV/BChE/hSA. Lane 6) was purified human serum BChE, positive control.

FIGURE 4 is a schematic depicting the generation of the pBCNN/BChE expression construct. SS = signal sequence. This expression construct provides for expression of recombinant BChE in the mammary gland of a transgenic mammal, and for the secretion of the recombinant BChE into the milk of a lactating transgenic mammal.

FIGURE 5 is a schematic depicting the exons and introns of the goat β-casein locus that are contained in the NotI linearized fragment of pBCNN/BChE. This BCNN-BChE fragment contains a BChE encoding sequence in place of goat β-casein locus sequences from the end of exon 2 through the majority of exon 7.

FIGURE 6 depicts a non-reducing BChE-activity gel of the whey phase of milk collected from BCNN-BChE transgenic mice. Whey phase samples were as follows: Lane 1) milk collected from BCNN-BChE transgenic mice; and Lanes 2 and 3) milk collected from non-trangenic mice (negative control). rBChE = recombinant BChE.

FIGURE 7 depicts a non-reducing BChE-activity gel of the whey phase of milk collected from BCNN-BChE transgenic goats. Whey phase samples were as follows: Lane 1) purified human serum BChE, positive control; Lane 2) milk from a non-transgenic goat, negative control; and Lanes 3-5) three independent milk samples collected from the same female transgenic goat.

FIGURE 8 depicts silver staining of a denaturing SDS-PAGE gel of recombinant BChE purified from milk collected from a BCNN-BChE transgenic goat. Samples were reduced in the presence of DTT prior to loading onto the gel. Samples were as follows: Lane 1) 0.2 µg of BChE purified from the milk of a BCNN-BChE transgenic goat; and Lane 2) 0.2 µg of purified human serum BChE, positive control.

FIGURE 9 is a schematic depicting the generation of the pWAP/BChE construct. This expression construct provides for expression of recombinant BChE in the mammary gland of a transgenic mammal, and for the secretion of the recombinant BChE into the milk of a lactating transgenic mammal

FIGURE 10 is a shematic depicting the linear NotI fragment of pWAP/BChE.

FIGURE 11 is a schematic depicting the strategy for generating the expression construct pUM/BChE. UM = uromodulin. SS = signal sequence. This expression construct will provide for expression of recombinant BChE in the kidney of a transgenic mammal, and for the secretion of the recombinant BChE into the urine of a transgenic mammal.

FIGURE 12 is a schematic depicting the strategy for generating the expression construct pUP11/BChE. UPII = uroplakin II. SS = signal sequence. This expression construct will provide for expression of recombinant BChE in the urothelium of a transgenic mammal, and for the secretion of the recombinant BChE into the urine of a transgenic mammal.

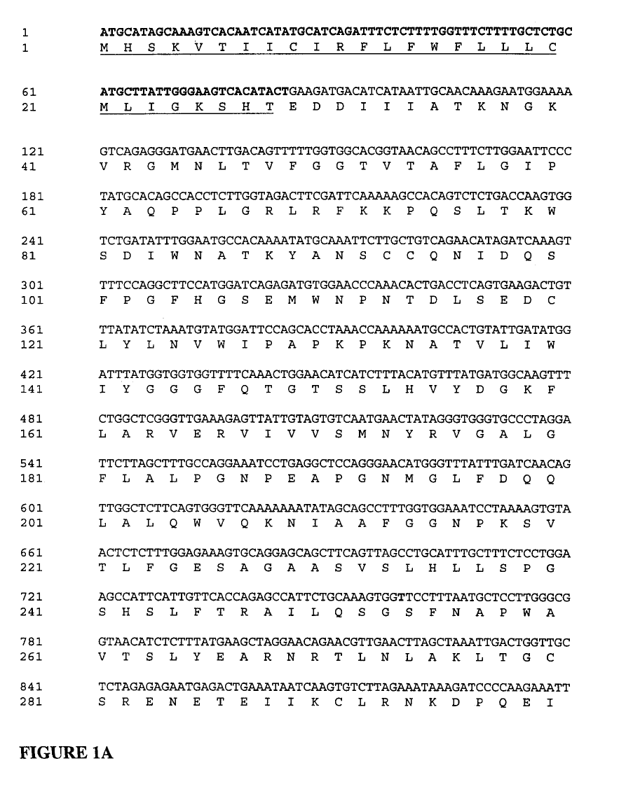

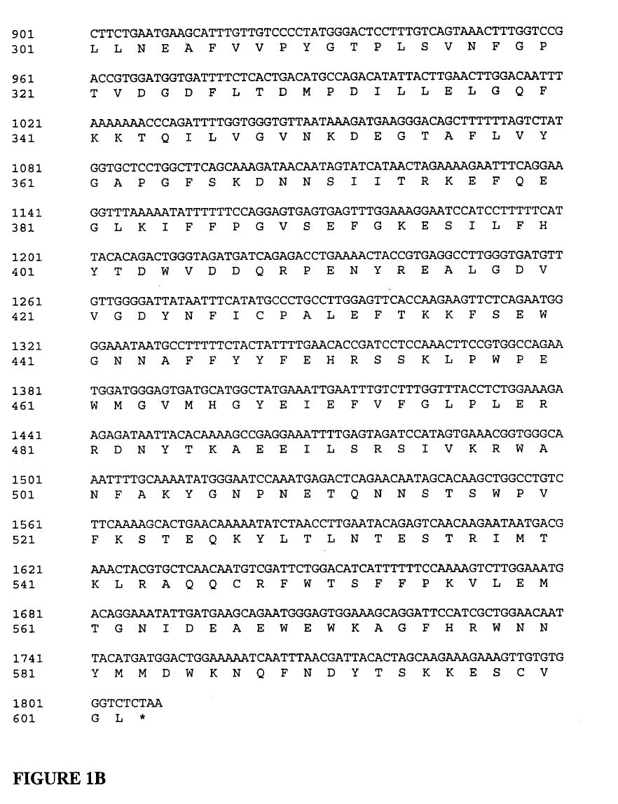

### DETAILED DESCRIPTION OF THE INVENTION

Definitions:

By "butyrylcholinesterase enzyme" or "BChE enzyme" is meant a polypeptide capable of hydrolizing acetylcholine and butyrylcholine, and whose catalytic activity is inhibited by the chemical inhibitor iso-OMPA. Preferred BChE enzymes to be produced by the present invention are mammalian BChE enzymes. Preferred mammalian BChE enzymes include human BChE enzymes. Most preferrably, the primary amino acid sequence of the BChE enzyme is subtantially identical to that of the native mature human BChE protein (As found in SEQ ID NO: 1). Such a BChE enzyme may be encoded by a nucleic acid sequence that is substantially identical identical to that of the native human BChE cDNA sequence (As found in SEQ ID NO: 2). The term "BChE enzyme" also encompasses pharmaceutically acceptable salts of such a polypeptide.

By "substantially identical" is meant a polypeptide or nucleic acid exhibiting at least 75%, preferably at least 85%, more preferably at least 90%, and most preferably at least 95% identity in comparison to a reference amino acid or nucleic acid sequence. For polypeptides, the length of sequence comparison will generally be at least 20 amino acids, preferably at least 30 amino acids, more preferably at least 40 amino acids, and most preferably at least 50 amino acids. For nucleic acids, the length of sequence comparison will generally be at least 60 nucleotides, preferably at least 90 nucleotides, and more preferably at least 120 nucleotides.

By "recombinant butyrylcholinesterase" or "recombinant BChE" is meant a BChE enzyme produced by a transiently transfected, stably transfected, or transgenic host cell or animal as directed by one of the expression constructs of the invention. The term "recombinant BChE" also encompasses pharmaceutically acceptable salts of such a polypeptide.

By "genetically-engineered DNA sequence" is meant a DNA sequence wherein the component sequence elements of the DNA sequence are organized within the DNA sequence in a manner not found in nature. Such a genetically-engineered DNA sequence may be found, for example, ex vivo as isolated DNA, in vivo as extra-chromosomal DNA, or in vivo as part of the genomic DNA.

By "expression construct" or "construct" is meant a nucleic acid sequence comprising a target nucleic acid sequence or sequences whose expression is desired, operably linked to sequence elements which provide for the proper transcription and translation of the target nucleic acid sequence(s) within the chosen host cells. Such sequence elements may include a promoter, a signal sequence for secretion, a polyadenylation signal, intronic sequences, insulator sequences, and other elements described in the invention. The "expression construct" or "construct" may further comprise "vector sequences." By "vector sequences" is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

By "bi-cistronic construct" is meant any construct that provides for the expression of two independent translated products. These two products may be translated from a single mRNA encoded by the bi-cistronic construct or from two independent mRNAs where each of the mRNAs is encoded within the same bi-cistronic construct. By "poly-cistronic construct" is meant any construct that provides for the expression of more than two independent translated products.

By "operably linked" is meant that a target nucleic acid sequence and one or more regulatory sequences ( e.g ., promoters) are physically linked so as to permit expression of the polypeptide encoded by the target nucleic acid sequence within a host cell.

By "signal sequence" is meant a nucleic acid sequence which, when incorporated into a nucleic acid sequence encoding a polypeptide, directs secretion of the translated polypeptide ( e.g. , a BChE enzyme and/or a glycosyltransferase) from cells which express said polypeptide. The signal sequence is preferably located at the 5' end of the nucleic acid sequence encoding the polypeptide, such that the polypeptide sequence encoded by the signal sequence is located at the N-terminus of the translated polypeptide. By "signal peptide" is meant the peptide sequence resulting from translation of a signal sequence.

By "mammary gland-specific promoter" is meant a promoter that drives expression of a polypedtide encoded by a nucleic acid sequence to which the promoter is operably linked, where said expression occurs primarily in the in the mammary cells of the mammal, wherefrom the expressed polypeptide may be secreted into the milk. Preferred mammary gland-specific promoters include the β-casein promoter and the whey acidic protein (WAP) promoter

By "urinary endothelium-specific promoter" is meant a promoter that drives expression of a polypedtide encoded by a nucleic acid sequence to which the promoter is operably linked, where said expression occurs primarily in the endothelial cells of the kidney, ureter, bladder, and/or urethra, wherefrom the expressed polypeptide may be secreted into the urine. The term "urothelium" or "urothelial cells" refers to the endothelial cells forming the epithelial lining of the ureter, bladder, and urethra.

By "host cell" is meant a cell which has been transfected with one or more expression constructs of the invention. Such host cells include mammalian cells in in vitro culture and cells found in vivo in an animal. Preferred in vitro cultured mammalian host cells include MAC-T cells and BHK cells.

By "transfection" is meant the process of introducing one or more of the expression constructs of the invention into a host cell by any of the methods well established in the art, including (but not limited to) microinjection, electroporation, liposome-mediated transfection, calcium phosphate-mediated transfection, or virus-mediated transfection. A host cell into which an expression construct of the invention has been introduced by transfection is "transfected". By "transiently transfected cell" is meant a host cell wherein the introduced expression construct is not permanently integrated into the genome of the host cell or its progeny, and therefore may be eliminated from the host cell or its progeny over time. By "stably transfected cell" is meant a host cell wherein the introduced expression construct has integrated into the genome of the host cell and its progeny.

By "transgene" is meant any segment of an expression construct of the invention which has become integrated into the genome of a transfected host cell. Host cells containing such transgenes are "transgenic." Animals composed partially or entirely of such transgenic host cells are "transgenic animals." Preferably, the transgenic animals are transgenic mammals ( e.g ., rodents or ruminants). Animals composed partially, but not entirely, of such transgenic host cells are "chimeras" or "chimeric animals."

Selection of BChE Enzymes

Butyrylcholinesterase derived from human serum is a globular, tetrameric molecule with a molecular mass of approximately 340 kDa. Nine Asn-linked carbohydrate chains are found on each 574-amino acid subunit. The tetrameric form of BChE is the most stable and is preferred for therapeutic purposes. Wildtype, variant, and artificial BChE enzymes can be produced by transgenic mammals according to the invention. BChE enzymes produced according to the instant invention have the ability to bind and/or hydrolyze organophosphate pesticides, war gases, succinylcholine, or cocaine.

Preferably, the BChE enzyme produced according to the invention comprises an amino acid sequence that is substantially identical to a sequence found in a mammalian BChE, more preferably, the BChE sequence is substantially identical to the human BChE. The BChE of the invention may be produced as a tetramer, a trimer, a dimer, or a monomer. In a preferred embodiment, the BChE of the invention has a glycosylation profile that is substantially similar to that of native human BChE.

In another preferred embodiment, the BChE enzyme produced according to the invention is fused to a human serum albumin (hSA) moiety. This fusion to hSA is expected to exhibit high plasma stability, and is expected to be either weakly or non-immunogenic for the organism in which it is used.

(a) Tetrameric BChE

The BChE produced according to the present invention is preferably in tetrameric form. It is believed that the tetrameric form of BChE is more stable and has a longer half-life in the plasma, thereby increasing its therapeutic effectiveness. BChE expressed recombinantly in CHO (Chinese hamster ovary) cells was found not to be in the more stable tetrameric form, but rather consisted of approximately 55% dimers, 10-30% tetramers and 15-40% monomers (Blong et al. , Biochem. J. (1997) 327:747-757). Recent studies have shown that a proline-rich amino acid sequence from the N-terminus of the collagen-tail protein caused acetylcholinesterase to assemble into the tetrameric form (Bon et al. , J. Biol. Chem. (1997) 272(5):3016-3021 and Krejci et al. , J. Biol. Chem. (1997) 272:22840-22847). Thus, to increase the amount of tetrameric BChE enzyme formed according to the invention, the DNA sequence encoding the BChE enzyme of the invention may comprise a proline-rich attachment domain (PRAD), which recruits recombinant BChE subunits ( e.g., monomers, dimers, and trimers) to form tetrameric associations. The PRAD preferably comprises at least six amino acid residues followed by a string of at least 10 proline residues. An example of a PRAD useful in the invention comprises the sequence (Glu-Ser-Thr-Gly ₃ -Pro ₁₀ ) (SEQ ID NO: 40). The PRAD may be included in a bi-cistronic expression construct which encodes both the PRAD and the BChE enzyme, or the PRAD and the BChE enzyme may be encoded in separate constructs. Alternatively, encoded PRAD may be attached directed to the encoded BChE enzyme. The invention also contemplates addition of a PRAD, which can be synthetic ( e.g., polyproline) or naturally occurring, to a mixture comprising recombinant BChE, to induce rearrangement of the BChE enzyme into tetramers.

(b) Non-tetrameric BChE

Although it is believed that tetrameric BChE will be the most therapeutically effective form of BChE for the treatment and/or prevention of organophosphate poisoning, other forms of the enzyme ( e.g., monomers, dimers, and trimers) have demonstrated substrate activity and are also encompassed by the invention. However, the observation that non-tetrameric forms of BChE are less stable in vivo does not rule out their usefulness in in vivo applications. Higher doses or more frequent in vivo administration of the non-tetrameric forms of BChE can result in satisfactory therapeutic activity.

The non-tetrameric forms of BChE are also useful in applications which do not require in vivo administration, such as the clean-up of lands used to store organophosphate compounds, as well as decontamination of military equipment exposed to organophosphates. For ex vivo use, these non-tetrameric forms of BChE may be incorporated into sponges, sprays, cleaning solutions or other materials useful for the topical application of the enzyme to equipment and personnel. These forms of the enzyme may also be applied externally to the skin and clothes of human patients who have been exposed to organophosphate compounds. The non-tetrameric forms of the enzyme may also find applications as barriers and sealants applied to the seams and closures of military clothing and gas masks used in chemical warfare situations.

(c) Fusion of BChE to human serum albumin

Another means of achieving plasma stability and longer half-life of recombinant BChE produced according to the invention is to provide a recombinantly produced BChE fused to human serum albumin (hSA). This fusion protein is believed to exhibit high plasma stability and an advantageous distribution in the body, and is expected to be either weakly or non-immunogenic for the organism in which it is used.

The BChE enzyme amino acid sequences and hSA amino acid sequences of the fusion protein may or may not be separated by linker amino acid sequences ( e.g. , a poly-glycine linker). Such linker amino acid sequences are often included to promote proper folding of the different domains of a fusion protein ( e.g. , hSA domain and BChE enzyme domain). By promoting proper folding of the BChE enzyme domain, such linker sequences may promote maintenance of catalytic activity.

For example, hSA may be fused to either the N-terminus or the C-terminus of BChE. In preferred embodiments the hSA moiety is fused to the C-terminal end of the BChE enzyme. This fusion is expected to provide a fusion protein that maintains BChE catalytic activity. In one embodiment for fusion of hSA to the N-terminal end of BChE, the plasmid pYG404 can be used, as described in EP 361,991. This plasmid contains a restriction fragment encoding the prepro-hSA gene. The BChE-encoding nucleic acid sequence can be amplified by PCR using primers that are exclusive of the termination codon and signal sequence. This BChE-encoding PCR product may be introduced at the 3' end of the pYG404 prepro-hSA sequence, in the same translational frame. In one embodiment for fusion of hSA to the C-terminal end of BChE, the hSA-encoding nucleic acid sequence, without its signal sequence, is fused in translational frame to the 3' end of the BChE-encoding nucleic acid sequence.

In another embodiment, purified recombinant BChE may be conjugated in vitro to a hSA polypeptide. Conjugation may be achieved by any appropriate chemical or affinity ligand method. Particularly useful are hSA and BChE polypeptides with monovalent affinity ligand modifications. For in vitro conjugation, each protein to be conjugated ( e.g ., hSA can be separately produced by recombinant methods and isolated to the necessary purity, followed by in vitro conjugation, prior to administration.

(d) BChE Glycosylation profile

Naturally occurring human serum BChE is highly glycosylated, containing approximately 31% carbohydrate by weight of protein (Saxena et al., Molec. Pharmacol. (1998) 53:112-122). The carbohydrate content of cholinesterases, including human BChE, generally comprises about 33-40% N-acetylglucosamine, 21-31% mannose, 18-21% galactose, and 15-18% sialic acid. It has been suggested that the relatively high stability of the globular tetrameric form of human plasma BChE may be associated with the capping of the terminal carbohydrate residues with sialic acid.

Mammalian cells used in recombinant protein synthesis have glycosylation capabilities, but if BChE is not normally expressed by these host cells, the glycosylation pattern of the recombinantly produced BChE may differ from that of the natural glycoprotein. Since BChE is a heavily glycosylated molecule, it is difficult for a recombinant host cell to modify it faithfully. Indeed, it has been shown that BChE produced in CHO cells had a lower sugar content than that found in the native human protein (Yuan et al., Acta Pharmacologica Sinica, (1999) 20:74-80).

As a means of producing recombinant BChE with a glycosylation profile that more closely resembles that of the native enzyme, the present invention is directed to transgenic animals that express both a BChE enzyme and one or more glycosyltransferases in their mammary glands and/or urinary endothelium, as well as cultured mammalian cells that express both a BChE enzyme and one or more glycosyltransferases. The presence of the glycosyltransferases in the intracellular secretory pathway of cells that are also expressing a secreted form of BChE catalyzes the transfer of glycan moieties to said BChE enzymes. The invention also encompasses addition of one or more glycosyltransferases to an in vitro reaction for the transfer of glycan moieties to a recombinant BChE produced by the transgenic animals or transfected mammalian cell lines of the invention. For example, recombinant BChE may be sialylated using the in vitro reaction conditions described in Chitlaru, et al. Biochem. J. (1998) 336:647-658. Thus, the glycosyltransferase which catalyzes transfer of glycans to the BChE enzyme may be expressed by the same cell that expresses the BChE enzyme, or the glycosyltransferase may be obtained from an external source and added to the recombinant BChE.

Most bioactive terminal sugars are attached to common core structures by "terminal" glycosyltransferases. When two terminal enzymes compete with each other, the ultimate carbohydrate structure is determined by the specificity of the enzyme that acts first. According to the present invention, a terminal or branching glycosyltransferase, which is not normally produced by the host cell, is introduced and "over-expressed" in the cell according to the methods described herein. The recombinantly produced glycosyltransferase will successfully compete with the endogenous enzymes, producing a recombinant BChE which has a glycosylation profile which more closely resembles that of the native enzyme. The methods of the invention alter the glycosylation capabilities of mammary, bladder, or kidney epithelial cells in order to control carbohydrate attachment on the secreted BChE. Carbohydrate moieties are commonly attached to asparagine, serine, or threonine residues.

The basic procedure involves introduction of an expression construct comprising a nucleic acid sequence encoding a glycosyltransferase enzyme operably linked to elements that allow expression of the glycosyltransferase enzyme in the tissue of interest. A second expression construct, one of the BChE-encoding expression constructs described herein, is also introduced. Alternatively, the BChE enzyme and the glycosyltransferase may be encoded in a single bi-cistronic construct. An example of a bi-cistronic construct of the invention would be a construct which comprises a WAP promoter; a nucleic acid sequence which encodes both a BChE enzyme and a glycosyltransferase, in which an IRES (internal ribosomal entry site) is included between the sequence encoding the BChE enzyme and the sequence encoding the glycosyltransferase; and signal sequences to provide secretion of the BChE enzyme and the glycosyltransferase. This construct may be introduced into the genome of a mammalian host cell by techniques well known in the art including microinjection, electroporation, and liposome-mediated transfection, calcium phosphate-mediated transfection, virus-mediated transfection, and nuclear transfer techniques. Accordingly, the recombinant BChE that is ultimately secreted by the host cell will have a more predictable glycosylation pattern. The invention also encompasses the generation of transgenic mammals that secrete a BChE enzyme and a glycosyltransferase in their milk and/or urine through cross-breeding of transgenic mammals that secrete a BChE enzyme only with transgenic mammals of the same species that secrete the desired glycosyltransferases, to produce transgenic mammals that secrete both enzymes.

The preferred glycosyltransferase enzymes for use in accordance with the present invention are sialyltransferases. Other enzymes that alter the glycosylation machinery whose production within a host cell may be desirable include fucosyltransferases, mannosyltransferases, acetylases, glucoronyltransferases, glucosylepimerases, galactosyltransferases, β-acetylgalactosaminyltransferases, N-acetylglucosaminyltransferases, and sulfotransferases. For a description of such transferases see, for example, Hennet, Cell Mol. Life Sci. (2002) 59:1081-1095; Harduin-Lepers et al. , Biochimie (2001) 83:727-737; and Takashima, et al. J. Biol. Chem. (2002) 277:45719-45728. Sequences that encode any one or more of such glycosyltransferases may be introduced into host cells according to the invention. These glycosyltransferases may be encoded in separate expression constructs, or included in any one or more bi-cistronic or poly-cistronic constructs. Thus, it should be noted that the invention allows for simultaneous expression in the milk and/or urine of a mammal of a BChE enzyme and one or more glycosyl transferases. The glycosyltransferases to be expressed are selected so as to effect transfer of one or more of the desired carbohydrate moieties to the BChE enzyme.

In the event that independent transcripts to encode the BChE enzyme and the respective glycosyltransferses, it is preferred that different promoters are used to express the different transcripts. For example, if the nucleic acid sequence encoding the BChE enzyme is operably linked to a mammary gland-specific casein promoter, it is preferred that nucleic acid sequence encoding the glycosyltransferase is operably linked to a different mammary gland-specific promoter, such as a WAP promoter. Although it is preferred to use different promoters in this instance, the invention also encompasses the use of the same promoter.

(e) Production of nucleic acid sequences which encode mutant BChE enzymes

The amino acid sequence of wildtype human BChE is set forth in U.S. Patent No. 6,001,625 to Broomfield et al., which is hereby incorporated herein in its entirety. This patent also discloses a mutant human BChE enzyme in which the glycine residue at the 117 position has been replaced by histidine (identified as G117H). This mutant BChE has been shown to be particularly resistant to inactivation by organophosphate compounds (Lockridge et al. , Biochemistry (1997) 36:786-795). Accordingly, this particular form of the BChE enzyme is especially useful for treatment of pesticide or war gas poisoning. Additional variants and mutants of BChE enzymes which may be produced according the methods of the present invention are disclosed in the U.S. Patent No. 6,001,625.

A number of methods are known in the art for introducing mutations within target nucleic acid sequences which may be applied to generate and identify mutant nucleic acid sequences encoding mutant BChE enzymes. Such mutant BChE enzymes may have altered catalytic properties, temperature profile, stability, circulation time, and affinity for cocaine or other substrates and/or certain organophosphate compounds; increased or decreased formation of BChE tetramers, dimers, or monomers; or other desired features. The mutant nucleic acid sequences encoding such mutant BChE enzymes may be used according to the present invention.

The template nucleic acid sequences to be used in any of the described mutagenesis protocols may be obtained by amplification using the PCR reaction (U.S. Patent Nos. 4,683,202 and 4,683,195) or other amplification or cloning methods. The described techniques can be used to generate a wide variatey of nucleic acid sequence alterations including point mutations, deletions, insertions, inversions, and recombination of sequences not linked in nature. Note that in all cases sequential cycles of mutation and selection may be performed to further alter a mutant BChE enzyme encoded by a mutant nucleic acid sequence.

Mutations can be introduced within a target nucleic acid sequence by many different standard techniques known in the art. Site-directed in vitro mutagenesis techniques include linker-insertion, nested deletion, linker-scanning, and oligonucleotide-mediated mutagenesis (as described, for example, in "Molecular Cloning: A Laboratory Manual," 2 ^{nd} Edition" Sambrook et al. , Cold Spring Harbor Laboratory (1989 and "Current Protocols in Molecular Biology," Ausubel et al. , eds. John Wiley & Sons (1989). Error-prone polymerase chain reaction (PCR) can be used to generate libraries of mutated nucleic acid sequences ( "Current Protocols in Molecular Biology" Ausubel et al. , eds. John Wiley & Sons (1989 and Cadwell et al. , PCR Methods and Applications (1992 2:28-33 ) . Altered BChE-encoding nucleic acid sequences can also be produced according to the methods of U.S. Patent No. 5,248,604 to Fischer. Cassette mutagenesis, in which the specific region to be altered is replaced with a synthetically mutagenized oligonucleotide, may also be used (Arkin et al. , Proc. Natl. Acad. Sci. USA (1992) 89:7811-7815; Oliphant et al. , Gene (1986) 44:177-183; Hermes et al. , Proc. Natl. Acad. Sci. USA (1990) 87:696-700). Alternatively, mutator strains of host cells can be employed to increase the mutation frequency of an introduced BChE encoding nucleic acid sequence (Greener et al. , Strategies in Mol. Biol. (1995) 7:32).

Another preferred method for generating and identifying mutant nucleic acid sequences encoding mutant BChE enzymes relies upon sequence or DNA "shuffling" to generate libraries of recombinant nucleic acid sequences encoding mutant BChE enzymes. The resultant libraries are expressed in a suitable host cell lines and screened for production of BChE enzymes with desired characteristics. For example, if a DNA fragment which encodes for a protein with increased binding efficiency to a ligand is desired, the BChE enzymes encoded by each of the sequence fragments of the library may be tested for their ability to bind to the ligand by methods known in the art (i.e., panning, affinity).

According to the "shuffling" technique, libraries of recombinant BChE-encoding nucleic acid sequences are generated from a population of related-nucleic acid sequences that comprise sequence regions having substantial sequence identity, and which can therefore be homologously recombined in vitro or in vivo . At least two species of BChE encoding nucleic acid sequences (for example, two nucleic acid sequence variants of human BChE) are combined in a recombination system suitable for generating a sequence-recombined library, where each nucleic acid sequence insert of the library comprises a combination of a portion of the first species of BChE-encoding nucleic acid sequence with at least one adjacent portion of another species of BChE-encoding nucleic acid sequence.

The DNA shuffling process for recombination and mutation is based upon random fragmentation of a pool of related nucleic acid sequences, followed by recombination of the fragments by primerless PCR in vitro or homologous recombination in vivo . The recombined products preferably contain a portion of each of the related nucleic acid sequences. The variant nucleic acid sequence species used are fragmented by nuclease digestion, partial extension PCR amplification, PCR stuttering, or other suitable fragmenting means. The resultant fragment may be recombined by PCR in vitro . Alternatively, the variant nucleic acid sequence species may be recombined in vivo . Preferably, combinations of in vitro and in vivo shuffling are performed. In one embodiment, the first plurality of selected library members is generated by a) in vitro fragmentation of variant nucleic acids sequence species , b) introduction of the resultant fragments into a host cell or organism, and c) in vivo homologous recombination of the fragments to form "shuffled" library members.

According to the invention, the variant nucleic acid sequences which may be "shuffled" to create and identify advantageous novel BChE-encoding nucleic acid sequences include, but are not limited to, nucleic acid sequences which encode taxonomically-related, structurally-related, and/or functionally-related enzymes and/or mutated variants thereof. The taxonomically-related sequences may comprise naturally occuring homologous nucleic acid sequences representing homologous genes from different species, homologous genes from the same species, or allelic variants of the same gene within a species. In this aspect, at least two naturally-occurring genes and/or allelic variants which comprise regions of at least 50 consecutive nucleotides which have at least 70 percent sequence identity, preferably at least 90 percent sequence identity, are selected from a pool of gene sequences, such as by hybrid selection or via computerized sequence analysis using sequence data from a database. The selected sequences are obtained as isolated nucleic acid sequences, either by cloning or via DNA synthesis, and shuffled by any of the various embodiments of the invention.

Naturally-Occuring Variants of Butyrylcholinesterase

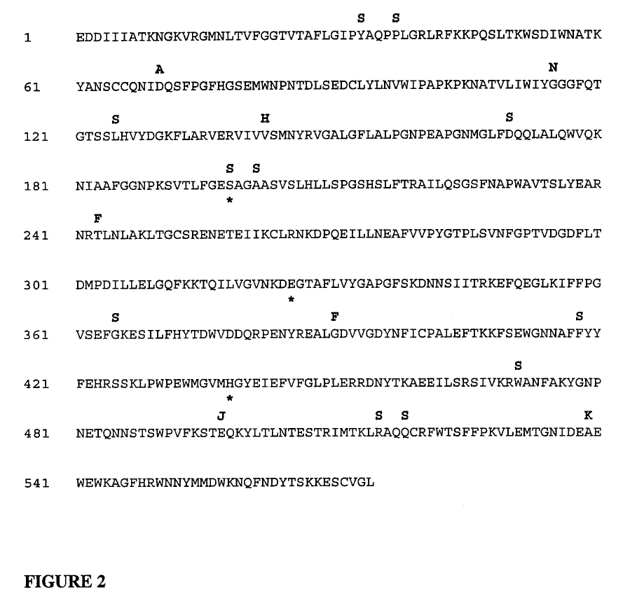

The BChE gene has four predominant allelic forms in humans, although 25 other forms responsible for various BChE genetic deficiencies are known (See Table 1 below, reproduced from the website of the American Society of Anesthesiologists, and FIGURE 2). The four predominant allelic forms are designated Eu, Ea, Ef, and Es. Eu is the wildtype, fully functional allele and carries the phenotype designation EuEu or UU. The Ea allele is referred to as atypical BChE. Phenotypically, the sera of persons homozygous for this gene (EaEa=AA) are only weakly active towards most substrates for ChE and show increased resistance to inhibition of enzyme activity by dibucaine. The Ef allele also gives rise to a weakly active enzyme, but exhibits increased resistance to fluoride inhibition. The Es gene (s for silent) is associated with absence of enzyme.

The mutations in the Ea and Ef gene products cause structural alterations in the active, site of the BChE enzyme resulting in less effective catalysis compared to the native (Eu) allele. Experimentally, these mutations result in the reduction in the binding affinity (increased Km) of competitive substrates. Clinically, the phenotypes that are most susceptible to prolonged succinylcholine-induced apnea are AA, SS, FF, FS, AS, AF, and UA.

Certain individuals carry an atypical BChE gene which functions normally to hydrolyze acetylcholine, but is unable to hydrolyze succinylcholine, a commonly used anesthetic. The most common variant with this problem is the atypical variant E ^{s} , for which 3-6% of the Caucasian population is heterozygous and about 0.05% is homozygous. Another variant, E ¹ , causes the complete absence of catalytically active serum BChE in homozygotes. This type of "silent" enzyme cannot hydrolyze any ChE substrate, nor can it bind organophosphate compounds. Individuals carrying atypical or silent BChE genes are subject to prolonged apnea following surgery in which succinylcholine is administered. High frequency of atypical and silent BChE genes has been reported among Iraqi and Iranian Jews (11.3% for heterozygotes and 0.08% for homozygotes). This could explain the high frequency of reports of prolonged apnea following surgery in Israel and apparently in many other other countries. Accordingly, a recombinant BChE may be administered to patients harboring these, or similar mutations, to alleviate or prevent prolonged post-surgical apnea.

TABLE 1
Structural Basis of Phenotype of Human BChE Variants

| | | |
|---|---|---|
| Variant | Effect of Mutation | Phenotype Alteration |
| Atypical | D70G^{‡} | Resistance to dibucaine |
| | | inhibition |
| Fluoride-resistant | T243M | Resistance to fluoride |
| | | inhibition |
| Fluoride-resistant | G390V | Resistance to fluoride |
| | | inhibition |
| K-variant | A539T | Activity reduced by 30% |
| J-variant | E497V | Activity reduced by 70% |
| H-variant | V142M | Activity reduced by 90% |
| Sc-variant | A184V | Decreased affinity for |
| | | succinylcholine |
| Silent-1 | Frameshift | No activity |
| | at codon 117 | |
| Silent-2 | Frameshift | No activity |
| | at codon 6 | |
| Silent-3 | Stop codon | No activity |
| | at codon 500 | |
| Silent-4 | P37S | No activity |
| Silent-5 | G365R | Trace activity |
| Silent-6 | Frameshift at | No activity |
| | at codon 315 | |
| Silent-8 | W471R | Trace activity |
| Silent-9 | D170E | No activity |
| Silent-10 | Q518L | Trace activity |
| Silent-11 | S198G | No activity |
| Silent-12 | Insertion of Alu | No activity |
| | element at | |
| | codon 355 | |
| Silent-13 | Altered splicing | No activity |
| | of intron 2 | |
| Silent-14 | L125F | Trace activity |
| Silent-16 | A201T | No activity |
| Silent-17 | Y33C | No activity |
| Silent-18 | Stop codon | No activity |
| | at codon 271 | |
| Silent-19 | F148S | Trace activity |
| Silent-20 | R515C | Trace activity |
| Silent-21 | Stop codon | No activity |
| | at codon 465 | |
| Unstable | G115D | Low, unstable |
| | | activity |

^{‡} Numbers represent residue position in the mature wild-type human BChE enzyme, once the signal peptide has been cleaved.

FIGURE 2 depicts the amino acid sequence of the mature wild-type human BChE enzyme and locations of altered residues in some BChE variants.

Assembly of Expression Constructs

The recombinant DNA methods employed in practicing the present invention are standard procedures, well-known to those skilled in the art (as described, for example, in "Molecular Cloning: A Laboratory Manual, 2 ^{nd} Edition," Sambrook et al. , Cold Spring Harbor Laboratory (1989, "A Practical Guide to Molecular Cloning," Perbal (1984, and "Current Protocols in Molecular Biology," Ausubel et al. , eds. John Wiley & Sons (1989). These standard molecular biology techniques can be used to prepare the expression constructs of the invention.

Expression constructs comprise elements necessary for proper transcription and translation of a target nucleic acid sequence within the chosen host cells, including a promoter, a signal sequence to provide secretion of the translated product, and a polyadenylation signal. Such expression constructs may also contain intronic sequences or untranslated cDNA sequences intended to improve transcription efficiency, translation efficiency, and/or mRNA stability. The nucleic acid sequence intended for expression may possess its endogenous 3' untranslated sequence and/or polyadenylation signal or contain an exogenous 3' untranslated sequence and/or polyadenylation signal. For example the promoter, signal sequence, and 3' untranslated sequence and polyandenylation signal of casein may be used to mediate expression of a nucleic acid sequence encoding BChE within mammary host cells. Codon selection, where the target nucleic acid sequence of the construct is engineered or chosen so as to contain codons preferentially used within the desired host cell, may be used to minimize premature translation termination and thereby maximize expression.

The inserted nucleic acid sequence may also encode an epitope tag for easy identification and purification of the encoded polypeptide. Preferred epitope tags include myc, His, and FLAG epitope tags. The encoded epitope tag may include recognition sites for site-specific proteolysis or chemical agent cleavage to faciliate removal of the epitope tag following protein purification. For example a thrombin cleavage site could be incorporated between the recombinant BChE and its epitope tag. Epitope tags may be fused to the N-terminal end or the C-terminal end of a recombinant BChE. Preferrably, the epitope tag is fused to the C-terminal end of a recombinant BChE; such C-terminal fusion proteins are expected to maintain cataytic activity and to retain the ability to oligomerize.

The expression constructs of the invention which provide expression of a BChE enzyme in the desired host cells may include one or more of the following basic components.

A) Promoter

These sequences may be endogenous or heterologous to the host cell to be modified, and may provide ubiquitous ( i.e ., expression occurs in the absence of an apparent external stimulus and is not cell-type specific) or tissue-specific (also known as cell-type specific) expression.

Promoter sequences for ubiquitous expression may include synthetic and natural viral sequences ( e.g ., human cytomegalovirus immediate early promoter (CMV); simian virus 40 early promoter (SV40); Rous sarcoma virus (RSV); or adenovirus major late promoter) which confer a strong level of transcription of the nucleic acid molecule to which they are operably linked. The promoter can also be modified by the deletion and/or addition of sequences, such as enhancers ( e.g., a CMV, SV40, or RSV enhancer), or tandem repeats of such sequences. The addition of strong enhancer elements may increase transcription by 10-100 fold.

For specific expression in the mammary tissue of transgenic animals, the promoter sequences may be derived from a mammalian mammary-specific gene. Examples of suitable mammary-specific promoters include: the whey acidic protein (WAP) promoter (U.S. Patent Nos. 5,831,141 and 6,268,545; Andres et al. , Proc. Natl. Acad. Sci. USA (1987) 84(5):1299-1303), αS1-casein (U.S. Patent Nos. 5,750,172 and 6,013,857, PCT Publication Nos. WO91/08216 and WO93/25567), αS2-casein, β-casein (U.S. Patent No. 5,304,489; Lee, et al. Nucleic Acids Res. (1988) 16:1027-1041), κ-casein (Baranyi et al. , Gene (1996) 174(1):27-34; Gutierrez et al. , Transgenic Research (1996) 5(4):271-279), β-lactoglobin (McClenaghan et al. , Biochem. J. (1995) 310(Pt2):637-641), and α-lactalbumin (Vilotte et al. , Eur. J. Biochem. (1989) 186: 43-48; PCT Publication No. WO88/01648).

For specific expression in the urinary endothelium of transgenic animals, the promoter sequences may be derived from a mammalian urinary endothelium-specific gene. Examples of suitable urinary endothelium-specific promoters include the uroplakin II promoter (Kerr et al. , Nature Biotechnology (1998) 16(1):75-79), and the uromodulin promoter (Zbikowska et al. , Biochem. J. (2002) 365(Pt1):7-11; Zbikowska et al. , Transgenic Res. 2002 11(4):425-435).

B) Intron Inclusion

Nucleic acid sequences containing intronic sequences ( e.g ., genomic sequences) may be expressed at higher levels than intron-less sequences. Hence, inclusion of intronic sequences between the transcription initiation site and the translational start codon, 3' to the translational stop codon, or inside the coding region of the BChE-encoding nucleic acid sequence may result in a higher level of expression.

Such intronic sequences include a 5' splice site (donor site) and a 3' splice site (acceptor site), separated by at least 100 base pairs of non-coding sequence. These intronic sequences may be derived from the genomic sequence of the gene whose promoter is being used to drive BChE expression, from a native BChE gene, or another suitable gene. Such intronic sequences should be chosen so as to minimize the presence of repetitive sequences within the expression construct, as such repetitive sequences may encourage recombination and thereby promote instability of the construct. Preferrably, these introns can be positioned within the BChE-encoding nucleic acid sequence so as to approximate the intron/exon structure of the native human BChE gene.

C) Signal Sequences

Each expression construct will additionally comprise a signal sequence to provide secretion of the translated recombinant BChE from the host cells of interest ( e.g., mammary or uroepithelial cells, or mammalian cell culture). Such signal sequences are naturally present in genes whose protein products are normally secreted. The signal sequences to be employed in the invention may be derived from a BChE gene, from a gene specifically expressed in the host cell of interest ( e.g ., casein or uroplakin gene), or from another gene whose protein product is known to be secreted ( e.g ., from human alkaline phosphatase, mellitin, the immunoglobulin light chain protein Igκ, and CD33); or may be synthetically derived.

D) Termination Region

Each expression construct will additionally comprise a nucleic acid sequence which contains a transcription termination and polyadenylation sequence. Such sequences will be linked to the 3' end of the BChE-encoding nucleic acid sequence. These sequences may comprise the 3' end and polyadenylation signal from the gene whose 5'-promoter region is driving BChE expression ( e.g., the 3' end of the goat β-casein gene). Alternatively, such sequences will be derived from genes in which the sequences have been shown to regulate post-transcriptional mRNA stability ( e.g. , those derived from the bovine growth hormone gene, the β-globin genes, or the SV40 early region).

E) Other features of the expression constructs

The BChE-encoding nucleic acid sequences of interest may be modified in their 5' or 3' untranslated regions (UTRs), and/or in regions coding for the N-terminus of the BChE enzyme so as to preferentially improve expression. Sequences within the BChE-encoding nucleic acid sequence may be deleted or mutated so as to increase secretion and/or avoid retention of the BChE enzyme product within the cell, as regulated, for example, by the presence of endoplasmic reticulum retention signals or other sorting inhibitory signals.

In addition, the expression constructs may contain appropriate sequences located 5' and/or 3' of the BChE-encoding nucleic acid sequences that will provide enhanced integration rates in transduced host cells ( e.g. , ITR sequences as per Lebkowski et al. , Mol. Cell. Biol. (1988) 8:3988-3996). Furthermore, the expression construct may contain nucleic acid sequences that possess chromatin opening or insulator activity and thereby confer reproducible activation of tissue-specific expression of a linked transgene. Such sequences include Matrix Attachment Regions (MARs) (McKnight et al. , Mol Reprod Dev (1996) 44(2):179-184 and McKnight et al. , Proc Natl Acad Sci USA (1992) 89:6943-6947). See also Ellis et al., PCT publication No.: WO95/33841 and Chung and Felsenfield, PCT Publication No.: WO96/04390.

The expression contructs further comprise vector sequences which facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli ; the SV40 origin of replication; an ampicillin, neomycin, or puromycin resistance gene for selection in host cells; and/or genes ( e.g ., dihydrofolate reductase gene) that amplify the dominant selectable marker plus the gene of interest. Prolonged expression of the encoded BChE enzyme in in vitro cell culture may be achieved by the use of vectors sequences that allow for autonomous replication of an extrachromosomal construct in mammalian host cells ( e.g ., EBNA-1 and oriP from the Epstein-Barr virus).

The expression constructs used for the generation of transgenic animals may be linearized by restriction endonuclease digestion prior to introduction into a host cell. In a variant of this method, the vector sequences are removed prior to introduction into host cells, such that the introduced linearized fragment is comprised solely of the BChE-encoding sequence, 5'-end regulatory sequences ( e.g ., the promoter), and 3'-end regulatory sequences ( e.g. , the 3' transcription termination and polyadenylation sequences), and any flanking insulators or MARs. A cell transformed with such a fragment will not contain, for example, an E. coli origin of replication or a nucleic acid molecule encoding an antibiotic-resistance protein ( e.g ., an ampicillin-resistance protein) used for selection of transformed prokaryotic cells.

In another variant of this method, the restriction digested expression construct fragment used to transfect a host cell will include a BChE-encoding sequence, 5' and 3' regulatory sequences, and any flanking insulators or MARs, linked to a nucleic acid sequence encoding a protein capable of conferring resistance to a antibiotic useful for selection of transfected eukaryotic cells ( e.g ., neomycin or puromycin).

Generation of transfected cell lines in vitro

The expression constructs of the invention may be transfected into host cells in vitro . Preferred in vitro host cells are mammalian cell lines including BHK-21, MDCK, Hu609, MAC-T (U.S. Patent No. 5,227,301), R1 embryonic stem cells, embryonal carcinoma cells, COS, or HeLa cells. Protocols for in vitro culture of mammalian cells are well established in the art (see for example, Animal Cell Culture: A Practical Approach 3 Edition, J. Masters, ed., Oxford University Press, and Basic Cell Culture, 2 Edition, Davis, J.M. ed., Oxford University Press (2002)). Techniques for transfection are well established in the art and may include electroporation, microinjection, liposome-mediated transfection, calcium phosphate-mediated transfection, or virus-mediated transfection (see, for example, Artificial self-assembling systems for gene delivery , . Felgner et al. , eds., Oxford University Press (1996); Lebkowski et al. , Mol. Cell Biol. 1988 8(10):3988-3996; "Molecular Cloning: A Laboratory Manual" 2 ^{nd} Edition, Sambrook et al. , Cold Spring Harbor Laboratory:1989; and "Current Protocols in Molecular Biology", Ausubel et al. , eds. John Wiley & Sons (1989). Where stable transfection of the host cell lines is desired, the introduced DNA preferably comprises linear expression construct DNA, free of vector sequences, as prepared from the expression constructs of the invention. Transfected in vitro cell lines may be screened for integration and copy number of the expression construct. For such screening, the genomic DNA of a cell line is prepared and analyzed by PCR and/or Southern blot.

Transiently and stably transfected cell lines may be used to evaluate the expression contructs of the invention as detailed below, and to isolate recombinant BChE and/or glycosyltransferase proteins. Where the expression construct comprises a ubiquitous promoter any of a number of established mammalian cell culture lines may be transfected. Where the expression construct comprises a tissue-specific promoter, the host cell line should be compatible with the tissue specific promoter ( e.g., uromodulin promoter containing expression constructs may be transfected into baby hamster kidney BHK-12 cells).

Stably transfected cell lines may also be used to generate transgenic animals. For this use, the recombinant proteins need not be expressed in the in vitro cell line.

Evaluation of Expression Constructs

Prior to the generation of transgenic animals using the expression constructs of the invention, expression construct functionality can be determined using transfected in vitro cell culture systems. Genetic stability of the expression constructs, degree of secretion of the recombinant protein(s), and physical and functional attributes of the recombinant protein(s) can be evaluated prior to the generation of transgenic animals.

Where the expression construct comprises a ubiquitous promoter any of a number of established mammalian cell culture lines may be transfected. Where the expression construct(s) comprises mammary gland or urinary endothelium-specific promoters, mammary epithelium and bladder cell lines can be transfected. For example, the hamster kidney cell line BHK-21 (C-13) (ATCC #CCl-10) (Sikri et al. , Biochem. J. (1985) 225:481-486) and the dog kidney cell line MDCK (ATCC #CCL-34) can be used to test the functionality of uromodulin promoter containing expression constructs. The human urothelium cell line Hu609 (Stacey et al. , Mol. Carcinog. (1990) 3:216-225) may used to test the functionality of uroplakin promoter containing expression constructs.

To determine if cell lines transfected with the BChE-encoding expression constructs of the invention are producing recombinant BChE, the media from transfected cell cultures can be tested directly for the presence of the secreted protein by Western blotting analysis using anti-BChE antibody (Monsanto, St. Louis, MO) or assessed using an activity assay (Ellman et al. , Biochem. Pharmacol. (1961) 7:88-95). Where a cell line is stably transfected and has been shown to produce catalyticaly active recombinant protein, the cell lines may be used for large scale culture and purification of the recombinant protein. Such cell lines may also be used in the generation of transgenic animals.

Generation of Transgenic Mammals

Protocols for the generation of non-human transgenic mammals are well established in the art (see, for example, Transgenesis Techniques, Murphy et al. , eds., Human Press, Totowa, New Jersey (1993); Genetic Engineering of Animals, A. Puhler, ed., VCH Verlagsgesellschaft, Weinheim, New York (1993); and Transgenic Animals in Agriculture, Murray et al. , eds. Oxford University Press). For example, efficient protocols are available for the production of transgenic mice ( Manipulating the Mouse Embryo 2 Edition Hogan et al. , Cold Spring Harbor Press (1994) and Mouse Genetics and Transgenics: A Practical Approach . Jackson and Abbott, eds., Oxford University Press (2000)), transgenic cows (U.S Patent No. 5,633,076), transgenic pigs (U.S. Patent No. 6,271,436), and transgenic goats (U.S. Patent No. 5,907,080). Preferred examples of such protocols are summarized below. It will be appreciated that these examples are not intended to be limiting, and that transgenic non-human mammals comprising the expression constructs of the invention, as created by these or other protocols, necessarily fall within the scope of the invention.

Transgenic animals may be generated using stably transfected host cells derived from in vitro transfection. Where said host cells are pluripotent or totipotent, such cells may be used in morula aggregation or blastocyst injection protocols to generate chimeric animals. Preferred pluripotent/totipotent stably transfected host cells include primordial germ cells, embryonic stem cells, and embryonal carcinoma cells. In a morula aggregation protocol, stably transfected host cells are aggregated with non-transgenic morula-stage embryos. In a blastocyst injection protocol, stably transfected host cells are introduced into the blastocoelic cavity of a non-transgenic blastocyst-stage embryo. The aggregated or injected embryos are then transferred to a pseudopregnant recipient female for gestation and birth of chimeras. Chimeric animals in which the transgenic host cells have contibuted to the germ line may be used in breeding schemes to generate non-chimeric offspring which are wholly transgenic.

In an alternative protocol, such stably transfected host cells may be used as nucleus donors for nuclear transfer into recipient oocytes (as per Wilmut et al. , Nature (1997) 385: 810-813). For nuclear transfer, the stably transfected host cells need not be pluripotent or totipotent. Thus, for example, stably transfected fetal fibroblasts can be used ( e.g., Cibelli et al. , Science (1998) 280: 1256-8 and Keefer et al. , Biology of Reproduction (2001) 64:849-856). The recipient oocytes are preferrably enucleated prior to transfer. Following nuclear transfer, the oocyte is transferred to a pseudopregnant recipient female for gestation and birth. Such offspring will be wholly transgenic (that is, not chimeric).

In another alternative protocol, transgenic animals are generated by direct introduction of expression construct DNA into a recipient oocyte, zygote, or embryo. Such direct introduction may be achieved by pronuclear microinjection (Wang et al. , Molecular Reproduction and Development (2002) 63:437-443), cytoplasmic microinjection (Page et al. , Transgenic Res (1995) 4(6):353-360), retroviral infection ( e.g., Lebkowski et al , . Mol. Cell Biol. (1988) 8(10):3988-3996), or electroporation ("Molecular Cloning: A Laboratory Manual. Second Edition" by Sambrook et al. , Cold Spring Harbor Laboratory: (1989).

For microinjection and electroporation protocols, the introduced DNA should comprise linear expression construct DNA, free of vector sequences, as prepared from the expression constructs of the invention. Following DNA introduction and any necessary in vitro culture, the oocyte, zygote, or embryo is transferred to a pseudopregnant recipient female for gestation and birth. Such offspring may or may not be chimeric, depending on the timing and efficiency of transgene integration. For example, if a single cell of a two-cell stage embryo is microinjected, the resultant animal will most likely be chimeric.

Transgenic animals comprising two or more independent transgenes can be made by introducing two or more different expression constructs into host cells using any of the above described methods.

The presence of the transgene in the genomic DNA of an animal, tissue, or cell of interest, as well as transgene copy number, may be confirmed by techniques well known in the art, including hybridization and PCR techniques.

Some of the transgenic protocols result in the production of chimeric animals. Chimeric animals in which the transgenic host cells have contributed to the tissue-type wherein the promoter of the expression construct is active ( e.g. , mammary gland for WAP promoter) may be used to characterize or isolate recombinant BChE and/or glucosyltransferase enzymes. More preferably, where the transgenic host cells have contibuted to the germ line, chimeras may be used in breeding schemes to generate non-chimeric offspring which are wholly transgenic.

Wholly transgenic offspring, whether generated directly by a transgenic protocol or by breeding of a chimeric animals, may be used for breeding purposes to maintain the transgenic line and to characterize or isolate recombinant BChE and/or glucosyltransferase enzymes. Where transgene expression is driven by a urinary endothelium-specific promoter, urine of transgenic animals may be collected for purification and characterization of recombinant enzymes. Where transgene expression is driven by a mammary gland-specific promoter, lactation of the transgenic animals may be induced or maintained, where the resultant milk may be collected for purification and characterization of recombinant enzymes. For female transgenics, lactation may be induced by pregnancy or by administration of hormones. For male transgenics, lactation may be induced by administration of hormones (see, for example, Ebert et al. , Biotechnology (1994) 12:699-702). Lactation is maintained by continued collection of milk from a lactating transgenic.

Purification of recombinant BChE

Recombinant BChE may be isolated from the culture medium of BChE-secreting transfected cells in vitro , from the milk of transgenic animals expressing BChE in mammary gland, or from the urine of transgenic animals expressing BChE in urinary endothelium using a procainamide affinity chromatography protocol (as described in Lockridge et al. , Biochemistry (1997) 36:786-795). For purification from culture medium, the medium is centrifuged or filtered to remove cellular debris prior to application to the procainamide column. The medium may also be concentrated by ultrafiltration. For purification from milk, tangential flow filtration clarification may be used to remove caseins and fat prior to application to the procainamide column. For purification from urine, the urine is first centrifuged to remove cell debris. Then the urine is diluted to reduce salt concentration, as measured by conductivity. The resulting solution is then applied to the column.

To provide enhanced purity of recombinant BChE, additional steps such as blue Sepharose CL-6B chromatography or ion exchange chromatography in combination with ammonium sulfate fractionation may be performed. Enzyme purity may be evaluated by reverse phase HPLC. Purified recombinant BChE may be separated on Sephacryl S-300 to distinguish the tetrameric and monomeric forms of the enzyme.

Assays to characterize BChE

The assays described here may be used to characterize variant BChEs as produced by the described mutagenesis protocols prior to expression construct assembly, and/or to characterize recombinant BChE collected from culture medium of transfected cells or from the milk or urine of transgenic animals. These assays allow for characterization of BChE enzyme activity, stability, structural characteristics, and in vivo function.

Various methods for in vitro BChE enzymatic activity assays are described in the art (for example, Lockridge and La Du, J. Biol. Chem. (1978) 253:361-366; Lockridge et al. , Biochemistry (1997) 36:786-795; Plattborze and Broomfield, Biotechnol. Appl. Biochem. (2000) 31:226-229; and Blong et al. , Biochem. J. (1997) 327:747-757). Samples can be tested for the presence of enzymatically active recombinant BChE by using the activity assay of Ellman (Ellman et al. , Biochem. Pharmacol. (1961) 7:88). Levels of BChE activity can be estimated by staining non-denaturing 4-30% polyacrylamide gradient gels with 2 mM echothiophate iodide as substrate (as described in Lockridge et al. , Biochemistry (1997) 36:786-795), where this method is a modification of the same assays using 2 mM butyrylthiocholine as substrate (from Karnovsky and Roots J. Histochem. Cytochem. (1964) 12:219). Using these methods, the catalytic properties of a BChE enzyme, including K ₘ , V ₘₐₓ , and k _{cat} values, may be determined using butyrylthiocholine or acetylthiocholine as substrate. Other methodologies known in the art can also be used to assess ChE function, including electrometry, spectrophotometry, chromatography, and radiometric methodologies.

Purified recombinant BChE may be separated on Sephacryl S-300 to distinguish the tetrameric and monomeric forms of the enzyme. Relative amounts of BChE tetramers, dimers, and monomers can also be estimated by staining non-denaturing 4-30% polyacrylamide gradient gels with 2 mM echothiophate iodide as substrate (as described in Lockridge et al. , Biochemistry (1997) 36:786-795). A panel of monoclonal antibodies may be used to characterize the functional domains of the recombinant BChE.

A competitive enzyme-linked immunosorbent assay (ELISA) may be used to quantitate the concentration of BChE protein in a sample. This assay is based on a poly-clonal rabbit anti-human BChE antibody coupled to biotin, where binding of the biotinylated antibody to immobilized BChE antigen is competitively inhibited by an added standard or the test sample. The amount of label-bound antibody is inversely related to the concentration of BChE in the test sample.

The recombinant BChE may be further characterized by standard techniques well known in the art, including N-terminal sequencing, determination of carbohydrate content (especially terminal sialic acid content), tryptic and carbohydrate mapping, and determination of in vitro stability. For example, the composition, distribution, and structure of monosaccharide and oligosaccharide moieties of the recombinant BChE may be analyzed as described in Saxena et al. , Biochemistry (1997) 36:7481-7489.

Potential clinical effectiveness of a recombinant BChE sample against organophosphate poisoning or cocaine toxicity can be assessed both in vitro and in vivo . For example, in vitro OPAH activities of the potential substrates soman, sarin and tabun can be measured in a pH stat using a solution of the test recombinant BChE. The activity of recombinant BChE against VX and echothiophate can be measured in a microtitre plate using a variation of the Ellman method, with the OP compound replacing the butyrylthioline as substrate. Enzyme-catalyzed hydrolysis of cocaine can be recorded on a temperature-equilibrated Gilford Spectrophotometer at 240 nm (Xie et al. , Mol. Pharmacol. (1999) 55:83-91).

The in vivo half life and protective effect versus organophosphate poisoning of a recombinant BChE sample may be assessed in animal models, such as rodents or primates (for example as in Raveh et al. , Toxicol. Applied Pharm. (1997) 145:43-53; Broomfield et al. , J. Pharmacol. Exp. Ther. (1991) 259:633-638; Brandeis et al. , Pharmacol. Biochem. Behav. (1993) 46:889-896; Ashani et al , Biochem. Pharmacol. (1991) 41:37-41; and Rosenberg et al. , Life Sciences (2002) 72:125-134). Peak blood BChE-level may be determined following intramuscular injection or recombinant BChE as described in Raveh et al. , Biochem. Pharmacol. (1993) 45(12):2465. Similarly, the in vivo half life and protective effect versus cocaine toxicity of a recombinant BChE sample may be assessed in animal models (for example, as in Hoffman et al. , J Toxicol Clin Toxicol (1996) 34:259-266 and Lynch et al , Toxicol. Appl. Pharmacol. (1997) 145:363-371).

Once the in vivo stability and efficacy of a recombinant BChE preparation has been verified in animal models, such preparations may be used for the treatment of various conditions, including organophopsate poisoning, post-surgical succinyl-choline induced apnea, or cocaine intoxication.

Treatment of Organophosphate Poisoning and Other Conditions

Exposure to organophosphate compounds can result in a wide variety of symptoms depending on the toxicity of the compound, the amount of compound involved in the exposure, the route of exposure, and the duration of the exposure. In mild cases, symptoms such as fatigue, weakness, dizziness, runny nose, bronchial secretions, nausea, and blurred vision may appear. In moderate cases, symptoms may include tightness in the chest, headache, sweating, tearing, drooling, excessive perspiration, vomiting, tunnel vision, and muscle twitching. In severe cases, symptoms include abdominal cramps, involuntary urination and diarrhea, muscular tremors, convulsions, staggering gait, pinpoint pupils, hypotension (abnormally low blood pressure), slow heartbeat, breathing difficulty, coma, and possibly death. Severe cases of organophosphate poisoning are observed after continued daily absorption of organophosphate pesticides, or from exposure to the most toxic organophosphate compounds used as chemical warfare agents. When symptoms of organophosphate poisoning first appear, it is generally not possible to tell whether a poisoning will be mild or severe. In many instances, when the skin is contaminated, symptoms can quickly go from mild to severe even though the area is washed. Some of the most toxic organophosphate compounds are those used as war gases. These compounds include tabun (GA), methyl parathion, sarin (GB), VX, soman (GD), diisopropylfluorophosphate, and PB. These compounds are easily absorbed through the skin, and may be inhaled or ingested. The symptoms of nerve gas poisoning are usually similar, regardless of the route of introduction.

Some of the most commonly used organophosphate pesticides include acephate (Orthene), Aspon, azinphos-methyl (Guthion), carbofuran (Furadan, F formulation), carbophenothion (Trithion), chlorfenvinphos (Birlane), chlorpyrifos (Dursban, Lorsban), coumaphos (Co-Ral), crotoxyphos (Ciodrin, Ciovap), crufomate (Ruelene), demeton (Systox), diazinon (Spectracide), dichlorvos (DDVP, Vapona), dicrotophos (Bidrin), dimethoate (Cygon, De-Fend), dioxathion (Delnav), disulfoton (Di-Syston), EPN, ethion, ethoprop (Mocap), famphur, fenamiphos (Nemacur), fenitrothion (Sumithion), fensulfothion (Dasanit), fenthion (Baytex, Tiguvon), fonofos (Dyfonate), isofenfos (Oftanol, Amaze), malathion (Cythion), methamidophos (Monitor), methidathion (Supracide), methyl parathion, mevinphos (Phosdrin), monocrotophos, naled (Dibrom), oxydemeton-methyl (Meta systox-R), parathion (Niran, Phoskil), phorate (Thimet), phosalone (Zolonc), phosmet (Irnidan, Prolate), phosphamidon (Dimecron), temephos (Abate), TEPP, terbufos (Counter), tetrachlorvinphos (Rabon, Ravap), and trichlorfon (Dylox, Neguvon).

Commonly used carbamate pesticides include aldicarb (Temik), bendiocarb (Ficam), bufencarb, carbaryl (Sevin), carbofuran (Furadan), formetanate (Carzol), methiocarb (Mesurol), methomyl (Lannate, Nudrin), oxamyl (Vydate), pirimicarb (pinmicarb, Pirimor) and propoxur (Baygon).

The present invention encompasses a method for the treatment of organophosphate poisoning comprising, administering to a subject in need thereof a therapeutically effective amount of recombinant BChE. The invention includes treatment of and amelioration of the symptoms resulting from exposure to organophosphate compounds, as well as methods of preventing symptoms of exposure to these compounds. Such methods involve administering to a subject an amount of recombinant BChE effective to protect against these symptoms, prior to exposure of the subject to an organophosphate compound.

The invention is also directed to methods for treating post-surgical, succinylcholine-induced apnea, and cocaine intoxication. These methods comprise administration to a subject suffering from post-surgical, succinylcholine-induced apnea or cocaine intoxication an effective amount of recombinant BChE.

EXAMPLES

Example 1. Production of Recombinant BChE in Cell Culture

1.1 Assembly of expression constructs

Standard recombinant DNA methods employed herein have been described in detail (see, for example, in "Molecular Cloning: A Laboratory Manual." 2 ^{nd} Edition" Sambrook et al. , Cold Spring Harbor Laboratory:1989, "A Practical Guide to Molecular Cloning" Perbal:1984, and "Current Protocols in Molecular Biology" Ausubel et al. , eds. John Wiley & Sons (1989). All DNA cloning manipulations were performed using E.coli STBII competent cells (Canadian Life Science, Burlington, Canada). Restriction and modifying enzymes were purchased from New England BioLabs (Mississauga, ON, Canada). All chemicals used were reagent grade and purchased from Sigma Chemical Co. (St. Louis, MO), and all solutions were prepared with sterile and nuclease-free WFI water (Hyclone, TX). Construct integrity was verified by DNA sequencing analysis provided by McMaster University (Hamilton, ON, Canada). Primers were synthesized by Sigma Genosys (Oakville, ON, Canada). PCR was performed using Ready-To-Go ^{TM} PCR beads (Pharmacia Biotech, Baie d'Urfé, PQ, Canada) or the High Fidelity PCR kit (Roche Diagnostics Canada, Laval, Canada).

In the expression contructs for the expression of recombinant BChE in in vitro cell culture, a sequence encoding human BChE was under the transcriptional control of a strong constitutive promoter and was linked to a signal sequence to provide secretion of the recombinant protein from the cells.

pCMV/IgKBChE

The human BChE cDNA was PCR amplified from a cDNA clone (ATCC #65726), with a sense primer Acb787 (5' AGA GAG GGG GCC CAA GAA GAT GAC ATC ATA ATT G 3') (SEQ ID NO: 3) containing an ApaI site (underlined) and a partial immunoglobulin kappa (Igκ) signal sequence, and an antisense primer Acb786 (5' CTG CGA GTT TAA ACT ATT AAT TAG AGA CCC ACA C 3') (SEQ ID NO: 4) including a PmeI site (underlined) and partial 3' sequence of the human BChE cDNA. The PCR product was digested with ApaI and PmeI, purified using GFX matrix (Pharmacia Biotech, Baie d'Urfé,PQ, Canada) and ligated into ApaI and PmeI digested pSecTag/MaSpI to generate pCMV/IgKBChE.

The construction of pSecTag/MaSpI is described in Lazaris et al. , Science (2002) 295: 472-476. Briefly, this plasmid contains the coding sequence of the spider silk protein gene MaSpI cloned into the vector pSecTag (Invitrogen). ApaI and PmeI digestion of pSecTag/MaSpI removes the MaSpI sequences as well as the His epitope tag sequences of the pSecTag vector. The remaining pSecTag vector sequences comprise the CMV promoter, the mouse IgK signal sequence, and bovine growth hormone termination and polyadenylation sequence.

The final expression construct pCMV/IgKBChE contains the sequence encoding mature human BChE, linked to the mouse Igκ signal sequence, under the transcriptional control of the cytomegalovirus promoter (CMV), as well as the bovine growth hormone termination and polyadenylation sequences for efficient transcription termination and transcript stability.

pCMV/BChE

pCMV/IgKBChE was digested with NheI and the ends were filled in using T4 DNA polymerase in the presence of dNTPs. This linearized vector then was digested with XbaI. This NheI (blunt-ended)-XbaI fragment was ligated to the BglII (blunt-ended)-XbaI fragment of the human BChE cDNA to generate pCMV/BChE, with BChE's own signal sequence retained. pCMV/BChE/hSA

PCR was performed using pCMV/BChE as a template with a sense primer Acb710 (5' GTG TAA CTC TCT TTG GAG AAA G 3') (SEQ ID NO: 5) containing a portion of 5' BChE sequence and an antisense primer Acb853 (5' TAT AAG TTT AAA CAT ATA ATT GGA TCC TCC ACC TCC GCC TCC GAG ACC CAC ACA ACT TTC TTT CTT G 3') (SEQ ID NO: 6) containing a PmeI site (underlined), a BamHI site (italic), a (Gly) ₆ -Ser linker (bolded) followed by a portion of 3' BChE sequence. The PCR product was digested with XbaI and PmeI, and ligated to XbaI and PmeI digested pCMV/BChE to generate pCMV/BChEmd.

PCR was performed using Marathon-ready human liver cDNA pool (Clontech) as a template with a sense primer Acb854 (5' ATA TAA GGA TCC GAT GCA CAC AAG AGT GAG GTT GCT CAT C 3') (SEQ ID NO: 7) containing a BamHI site (underlined) and partial sequence from the hSA cDNA 5' end (Genbank V00495, without the signal sequence), and an antisense primer Acb855 (5' ATT TAA GTT TAA ACT CAT TAT AAG CCT AAG GCA GCT TGA CTT GC 3') (SEQ ID NO: 8) including a PmeI site (underlined) and partial sequence from the hSA cDNA 3' end. This PCR product was digested with BamHI and PmeI and inserted into BamHI and PmeI digested pCMV/BChEmd to generate the final construct, pCMV/BChE/hSA. This expression construct encodes a BChE-hSA fusion protein.

1.2. Transfection and selection of stable cell lines.

Preparation of expression constructs for transfection:

The constructs pCMV/IgKBChE and pCMV/BChE/hSA were digested with FspI, and the resultant FspI-digested linear DNA, was prepared and used for transfection. Briefly, circular expression construct DNA was purified by the cesium chloride gradient technique. This purified DNA was restricted with FspI, precipitated, and resuspended in sterile deionized water.

Stably transfected MAC-T cell lines expressing recombinant BChE:

MAC-T cells (ATCC #CRL 10274, US Patent No. 5,227,301) were seeded at a density of 5 X 10 ⁵ cells per 100 mm dish. On the following day, cells were transfected with Lipofectamine PLUS Reagent (Invitrogen) as per the manufacturer's recommendations with 4 μg of the linearized pCMV/IgKBChE construct. Briefly, the DNA was diluted to a final volume of 750 μL with DMEM (Invitrogen) and 20 μL of PLUS Reagent was added to the mixture. The Lipofectamine was diluted to a final volume of 750 μL with DMEM. After incubation at ambient temperature for 15 min, the Lipofectamine and DNA mixtures were combined and complexes allowed to form for 15 min at room temperature.

The lipid-DNA complex mixture was applied to the cells, and the cells allowed to incubate for 3 hrs at 37 ^{o} C under 5% CO ₂ . The cells were then cultured for another 24 hrs in fresh medium containing 20% fetal bovine serum (FBS, Invitrogen). Subsequently, stably transfected cells were selected in DMEM containing 10% FBS, 5 μg/ml insulin (Sigma), and 100 μg/ml hygromycin B (Invitrogen). Colonies surviving selection were picked 7 to 14 days following transfection and expanded further.

The level of BChE activity in cell culture media from pCMV/IgKBChE transfected MAC-T cells was evaluated by measuring butyrylthiocholine iodide hydrolysis (see Ellman et al. , Biochem. Pharmacol. (1961) 7:88) using a commercially available test (Sigma). The assay was performed according to the manufacturer's recommendations. The resulting activity values in units/ml were converted to mg of active BChE by using the relationship: 1 mg of active BChE = 720 units. From over 100 clones tested, the one demonstrating the highest BChE activity, as tested by the Ellman activity assay was further evaluated in roller bottles containing serum-free DMEM. The amount of BChE activity under these conditions was estimated at 0.56 units per million cells (U/10 ⁶ ) per 24 hours.

A master cell bank was generated and used to initiate a hollow fiber bioreactor production run (Biovest, CP2500 model). Hollow fiber production of stable transfectants was established for large-scale production of recombinant BChE.

Stably transfected MAC-T cell lines expressing a recombinant BChE-hSA fusion:

MAC-T cells were seeded at a density of 2.5 X 10 ⁵ cells per 100 mm dish. On the following day, cells were transfected with Lipofectamine Reagent (Invitrogen) with 10 μg of the linearized pCMV/BChE/hSA construct. Briefly, the DNA was diluted to a final volume of 500 μL with DMEM (Invitrogen) and 60 μL of Lipofectamine was diluted to a final volume of 500 μL with DMEM. The two solutions were combined, vortexed for 10 sec and the complexes were allowed to form at room temperature for 30 min. DMEM was added to the lipid-DNA mixture up to a final volume of 5 ml. The mixture was then applied to the cells and allowed to incubate overnight at 37 ^{o} C under 5% CO ₂ . The cells were then cultured for another 24 hrs in DMEM containing 10% FBS, 5 μg/ml insulin (Sigma).

Stably transfected cells were selected in DMEM containing 10% FBS, 5 μg/ml insulin (Sigma), and 100 μg/ml hygromycin B (Invitrogen). Colonies surviving selection were picked 7 to 14 days following transfection and expanded further.

The level of BChE activity in cell culture media from pCMV/BChE/hSA transfected MAC-T cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.17 units per million cells (U/10 ⁶ ) per 24 hours. Thus, it was successfully demonstrated that the recombinant BChE-hSA fusion protein is active.

Stably transfected BHK cell lines expressing a recombinant BChE-hSA fusion:

These lines were generated using the same procedure for stable transfection of MAC-T cells with pCMV/BChE/hSA, with the exception that the cells were BHK (Baby Hamster Kidney) cells (supplied by Dr. G. Matleshewski of McGill University, also available from the ATCC, clone #CCl-10) and the selection media contained DMEM with 10% FBS and 300 μg/ml hygromycin B (Invitrogen). Colonies surviving selection were picked 7 to 14 days following transfection and expanded further.

The level of BChE activity in cell culture media from pCMV/BChE/hSA transfected BHK cells was evaluated using a commercially available test (Sigma). From over 100 clones tested, the one demonstrating the highest BChE activity was further evaluated in roller bottles containing serum-free DMEM. The amount of BChE activity under these conditions was estimated at 0.73 units per million cells (U/10 ⁶ ) per 24 hours.

1.3. Detection of recombinant BChE in culture media of transfected cells.

Western blotting analysis of non-denaturing PAGE gels and denaturing SDS-PAGE gels was used to detect the presence of recombinant BChE in cell culture media. Cell culture media from pCMV/IgKBChE transfected MAC-T cells, and pCMV/BChE/hSA transfected MAC-T or BHK cells, was electrophoresed on non-denaturing and denaturing pre-cast 4 - 20 %TRIS-glycine gels (Invitrogen). The samples were then transferred by electroblotting onto nitrocellulose membranes (Bio-Rad). Recombinant BChE on the membranes was detected using rabbit polyclonal antibodies raised against BChE (DAKO) at a dilution of 1:1000 and goat anti-rabbit horseradish peroxidase conjugated second antibody. Detection was performed according to manufacturer's protocol for enhanced chemiluminescence (ECL) detection (Amersham Pharmacia).

In such analyses, the anti-BChE antibodies specifically detected a protein of the appropriate molecular weight in cell culture media from transfected cells. These results confirmed the production of recombinant BChE, and of the recombinant BChE-hSA fusion protein, in transfected cell lines in in vitro culture.

1.4. BChE-activity gels

20 μL of samples of cell culture media from pCMV/IgKBChE transfected MAC-T cells, and pCMV/BChE/hSA transfected MAC-T and BHK cells, was electrophoresed on native 4-20% pre-cast TRIS-glycine gels at 100-125 V overnight and at 4°C. The gels were then stained for BChE activity with 2 mM of butyrylthiocholine iodide according to the Karnovsky and Roots method (Karnovsky and Roots, Histochem. Cytochem. (1964) 12:219-221). The staining procedure was performed at ambient temperature for two to six hours until the active protein bands were revealed.

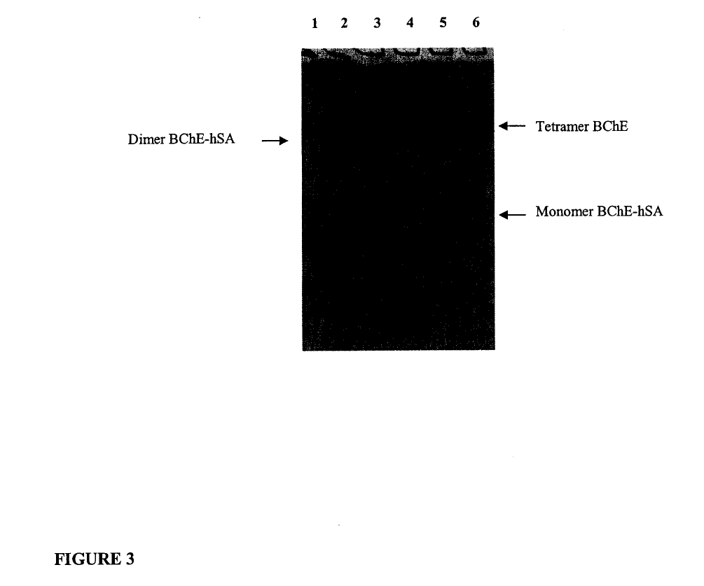

Conditioned media from pCMV/IgKBChE transfected MAC-T cells showed an active protein, migrating at the molecular weight size of a tetramer (FIGURE 3, lane 2). Conditioned media from MAC-T cells transfected with pCMV/BChE/hSA also showed expression of an active tetramer, as well as of active monomers and dimers (FIGURE 3, lane 3). Conditioned media from BHK cells transfected with pCMV/BChE/hSA showed high level expression of both an active monomer and an active dimer (FIGURE 3, lane 5)

The finding that MAC-T cells produce recombinant BChE predominantly in tetramer form is unexpected. In prior reports of recombinant expression of BChE in in vitro cultured cells, the tetrameric form was the least abundant ( e.g. , Blong et al. , Biochem. J. (1997) 327:747-757). Thus, the present invention provides for dramatically improved yields of tetrameric BChE enzyme (at least 50% of the produced BChE enzyme) using MAC-T cells transfected with the expression constructs of the invention.

This result also confirms that the recombinant BChE-hSA fusion protein is catalytically active, and may assemble into the dimeric form.

Example 2. Production of Recombinant Human BChE in Transgenic Mice

2.1. Expression construct pBCNN/BChE

In this expression construct, the BChE-encoding sequence is under the transcriptional control of a strong β-casein promoter to direct expression of recombinant BChE in the mammary gland, and linked to a β-casein signal sequence to direct secretion of recombinant BChE into milk produced by the mammary gland.

pUC18/BCNN

The goat β-casein promoter, including sequences through exon 2, were reverse PCR amplified from a genomic DNA library (SphI restriction digest) generated using goat blood (Clontech Genome Walking Library), using primers ACB582 (5'CAG CTA GTA TTC ATG GAA GGG CAA ATG AGG 3') (SEQ ID NO: 41) and ACB591 (5' TAG AGG TCA GGG ATG CTG CTA AAC ATT CTG 3') (SEQ ID NO: 42). The 6.0 kb product was subcloned into the pUC18 vector (Promega) and designated pUC18/5'bCN.

A 4.5 kb DNA fragment spanning exon 7 and the 3' end of the goat β-casein gene was reverse PCR amplified from the same library (BglII restriction digest) using primers ACB583 (5' CCA CAG AAT TGA CTG CGA CTG GAA ATA TGG 3') (SEQ ID NO: 43) and ACB601 (5' CTC CAT GGG TAA GCC TAA ACA TTG AGA TCT 3') (SEQ ID NO: 44). The fragment was subcloned in the pUC18 vector as designated pUC18/3'bCN.

The 4.3 kb fragment encompassing exon 7 and the 3' end of the goat β-casein gene was then PCR amplified from pUC18/3'bCN, using primer ACB620 (5' CTT TCT CAG CCC AAA GTT CTG CCT GTT C 3') (SEQ ID NO: 45), which introduces NotI and XhoI sites and primer ACB621 (5' CAA GTT CTC TCT CAT CTC CTG CTT CTC A 3') (SEQ ID NO: 46), which introduces SalI and NotI sites. This fragment was subcloned into the pUC18 vector and designated pUC18bCNA.

A 4.9 kb fragment containing the 5' end of the β-casein promoter including sequences through exon 2 was PCR amplified from pUC18/5'bCN using primer ACB618 (5' CAG TGG ACA GAG GAA GAG TCA GAG GAA G 3') (SEQ ID NO: 47), which introduces a BamHi and SacI site at the 5'end and primer ACB619 (5' GTA TTT ACC TCT CTT GCA AGG GCC AGA G 3') (SEQ ID NO: 48), which is near the starting ATG codon and introduces a XhoI site. This fragment was then subcloned into the pUC18bCNA expression vector by digesting with XhoI, which digests at the 5' end of the 3' bCN fragment and BamHI, which is present in the pUC18 vector just upstream of the XhoI site. This ligation generates the final pUC18/BCNN construct, which contains the β-casein promoter, including sequences up to exon 2, followed by an XhoI site, exon 7 and the 3' end of the β-casein gene.

pBCNN/BChE

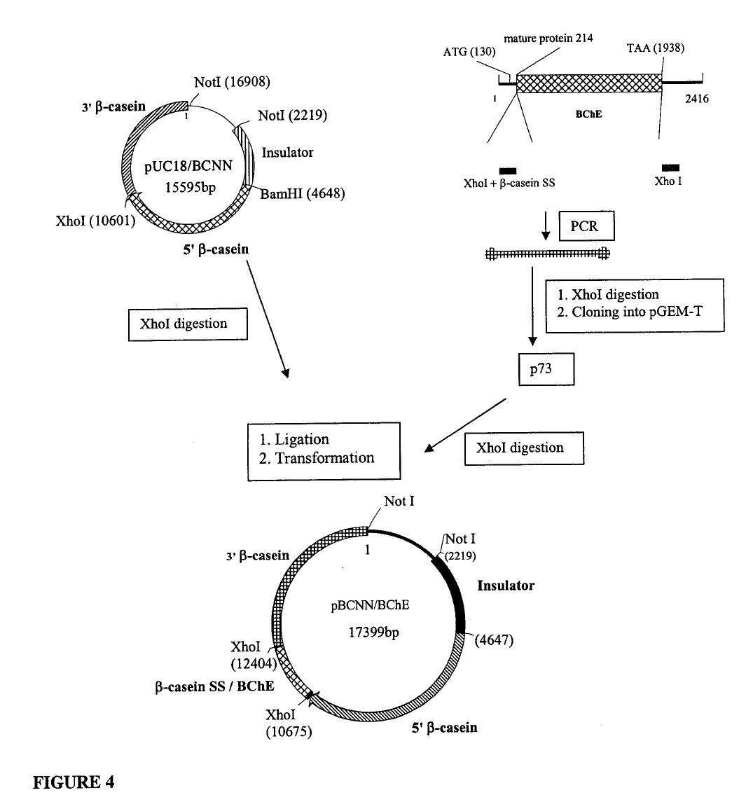

The human BChE cDNA was PCR amplified from a cDNA clone (ATCC #65726) with a sense primer Acb719 (5' ATA TTC TCG AG A GCC ATG AAG GTC CTC ATC CTT GCC TGT CTG GTG GCT CTG GCC CTT GCA AGA GAA GAT GAC ATC AT 3') (SEQ ID NO: 9) containing an XhoI restriction endonuclease site (underlined), goat β-casein signal sequence (italic), and a partial human BChE sequence; and an antisense primer, Acb718 (5' CTA TGA CTC GAG GCG ATC GCT ATT AAT TAG AGA CCC ACA C 3') (SEQ ID NO: 10) containing an XhoI site (underlined) and partial 3' human BChE sequence. The BChE PCR product was XhoI digested and subcloned into pGEM-T easy vector (Promega), to give the construct named p73. The BChE insert of p73 was excised by digestion with XhoI, purified with GFX matrix (Pharmacia Biotech, Baie d'Urfé, PQ, Canada) and ligated with XhoI-digested pUC18/BCNN to generate pBCNN-BChE. The generation of pBCNN/BChE is shown schematically in FIGURE 4.

pBCNN/BChE was digested with NotI, and the resultant NotI-digested linear DNA, free of bacterial sequences, was prepared and used to generate transgenic mice. Briefly, circular expression construct DNA was purified by the cesium chloride gradient technique. This purified DNA was restricted with NotI, electrophoresed, and the linear DNA fragment was gel purified. The DNA fragment was then mixed with cesium chloride and centrifuged at 20°C, 60,000 rpm for 16 to 20 hrs in a Beckman L7 ultracentrifuge using a Ti70.1 rotor (Beckman Instruments, Fullerton, Calif., USA). The DNA band was removed, dialyzed against WFI water for 2-4 hrs, and precipitated in ethanol. The precipitated DNA was resuspended in injection buffer (5 mM Tris pH 7.5, 0.1 mM EDTA, 10 mM NaCl) and dialyzed against the same buffer at 4°C for 8 hrs. Two additional dialysis steps were performed, one for 16 hrs and the second for at least 8 hrs. After dialysis the DNA was quantitated using a fluorometer. Prior to use an aliquot was diluted to 2-3 ng/ml in injection buffer.

As a result of this preparation, the linear BCNN/BChE fragment used to generate transgenic animals contained, in this order:

Dimerized chicken β-globin gene insulator;

Goat beta-casein promoter;

β-casein exon 1;

β-casein intron 1;

Partial β-casein exon 2;

XhoI cloning site;

β-casein signal sequence;

BChE-encoding sequence;

A STOP codon;

Partial β-casein exon 7;

β-casein intron 7;

β-casein exon 8;

β-casein intron 8;

β-casein exon 9; and

Additional β-casein 3' genomic sequence.

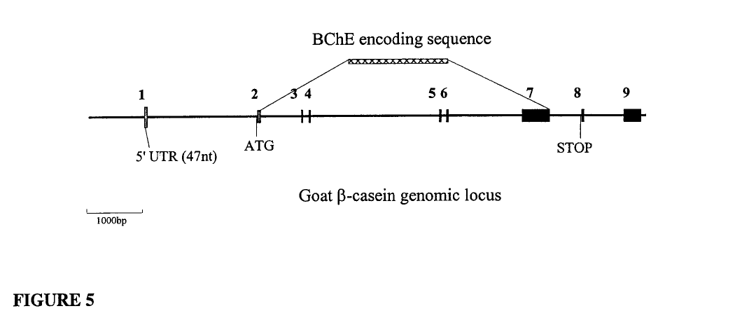

A schematic depicting the exons and introns of the goat β-casein locus that are contained in this fragment is shown in FIGURE 5.

2.2. Production of founders and subsequent generations of transgenic mice.

The production and maintenance of transgenic mice were conducted at the McIntyre Transgenic Core Facility of McGill University. Transgenic mice were generated by pronuclear microinjection essentially as described in Hogan et al. , "Manipulating the Mouse Embryo: A Laboratory Manual," Cold Spring Harbor Laboratory (1986). The BCNN/BChE linear fragment was microinjected into 414 fertilized eggs (strain FVB) and 22 pups were born.

At 2-3 weeks of age tail biopsies were taken, under anesthesia and DNA was prepared according to standard procedures well known to those skilled in the art, and described in detail, for example, in "Molecular Cloning: A Laboratory Manual, 2 ^{nd} Edition", Sambrook et al. , Cold Spring Harbor Laboratory (1989). The presence of the transgene in genomic DNA was confirmed by PCR and/or Southern analysis as described in Section 2.3., Identification of transgenic mice , below. Out of 28 tail DNA samples, 2 dead pup and 4 live founders (2 males and 2 females) were confirmed transgene positive. Southern analysis was also used to estimate transgene copy number.

Transgenic founder mice were bred with wild-type mice of the same strain for the generation of subsequent transgenic generations. One founder female has been used to establish a transgenic line with ~10 copies of the transgene. The other female and one of the male founders have been used to establish a trasgenic line with ~40 copies of the transgene. As shown in Table 2, the transgene was stably transmitted for 2 generations.

2.3. Identification of transgenic mice.

PCR analysis:

Genomic DNA purified from tail biopsies was quantitated by fluorimetry and PCR screened using three different primer sets. PCR was performed with the Ready-To-Go™ PCR beads (Pharmacia Biotech). Upon amplification the samples were analysed for the presence of the PCR product by electrophoresis on a 2% agarose gel. The quality of the DNA used in these PCR reactions was confirmed by the presence of the expected fragment of the endogenous mouse β-casein gene.

Primer set A, ACB712 (5' CTT CCG TGG CCA GAA TGG AT 3') (SEQ ID NO: 11) and ACB244 (5' CAT CAG AAG TTA AAC AGC ACA GTT AGT 3') (SEQ ID NO: 12), amplifies a 495 bp fragment from the 3' end of the transgene spanning the junction of the BChE and 3' genomic β-casein sequences.

Primer set B, ACB268 (5' AGG AGC ACA GTG CTC ATC CAG ATC 3') (SEQ ID NO: 13) and ACB659 (5' GAC GCC CCA TCC TCA CTG ACT 3') (SEQ ID NO: 14), amplifies a 893 bp fragment of the insulator sequence located at the 5' end of the transgene.

Primer set C, ACB572 (5' TTC CTA GGA TGT GCT CCA GGC T 3') (SEQ ID NO: 15) and ACB255 (5' GAA ACG GAA TGT TGT GGA GTG G 3') (SEQ ID NO: 16) amplifies a 510 bp portion of an endogenous mouse β-casein gene. This primer set serves as in internal positive control to indicate that the extracted DNA can be amplified by PCR.

Southern Blotting analysis:

Confirmation of transgene presence, and estimation of transgene copy number, was performed using Southern blotting analysis with Boehringer Mannheim's DIG system. Genomic DNA (5μg) extracted from tail biopsies was digested with XmeI and ApaLI. This digestion was followed by gel electrophoresis and Southern transfer to nylon membranes (Roche Diagnostics Canada). The blot was hybridized in a DIG Easy Hyb ^{™} buffer (Roche Diagnostics Canada) at 42°C overnight using an insulator probe labeled by the PCR DIG probe synthesis kit (Roche Diagnostics Canada), which hybridizes at the 5' end of the transgene. This insulator probe was PCR amplified from the pBCNN/BChE construct using the primers Acb266 (5' TGC TCT TTG AGC CTG CAG ACA CCT 3') (SEQ ID NO: 17) and Acb267 (5' GGC TGT TCT GAA CGC TGT GAC TTG 3') (SEQ ID NO: 18). The membrane was washed, detected by the CDP-Star™ substrate (Roche Diagnostics Canada) and visualized by the FluorChem™ 8000 System (Alpha Innotech Corporation). The size of the genomic DNA fragment detected by this probe varies depending on the site of integration.

The same membrane was stripped with stripping buffer (Roche Diagnostics Canada) and re-hybridized with a DIG-labeled PCR probe hybridizing within the BChE sequence. The probe was PCR amplified from the pBCNN/BChE construct using the primers Acb710 (5' GTG TAA CTC TCT TTG GAG AAA G 3') (SEQ ID NO: 5) and Acb819 (5' CCA GAG GTA AAC CAA AGA C 3') (SEQ ID NO: 19). This 725bp BChE-encoding sequence probe detects an 11 kb band of the transgene.

Upon analysis, the expected size bands were detected for all transgenic offspring and copy number was estimated. Transgene copy number has been stable for at least two generations (see Table 3). For example, the founder transgenic male (F0) with ~40 copies of the transgene has transmitted ~40 copies to all of his offspring (F1).

2.4. Analysis of recombinant BChE in the milk transgenic mice

Lactating female mice were milked after induction with an intraperitoneal injection of 5 i.u. of oxytocin.

The milking apparatus is described online (http://www.invitrogen.com/Content/Tech-online/molecular_biology/manuals_pps/pbc1_man.pdf). The amount of milk that was obtained varied from 50 - 100 µl. The milk was centrifuged at 3000 x g for 30 minutes at 4 ^{o} C, and the resultant whey phase was separated from the fat phase and precipitates. The whey phase was stored at -20 ^{o} C until analysis.

The milk was analyzed for BChE activity levels using the Ellman Assay, and for oligomerization of recombinant BCHE by analysis on non-denaturing activity gels. It is important to note that mouse milk contains endogenous levels of BChE activity that were controlled for in performing the activity assays. The non-denaturing activity gels showed a unique band for the endogenous mouse BChE that did not co-migrate with the recombinant BChE.

Levels BChE activity measured using the Ellman Assay

The Ellman BChE activity assay was performed on the whey phase of milk collected from transgenic mice. The whey phase of milk from 2 wild type FVB mice served as negative controls, while a partially purified human plasma BChE sample served as a standard. Samples were added in 100 μl of 0.1 M potassium phosphate buffer (pH 8.0) into each well of duplicate 96-well plates. 50 μl of DTNB reaction buffer were added into each well, and then mixed well. The plate was incubated at room temperature for 10 minutes. Absorbance of the plate at 405 nm was measured with Vmax ^{®} Kinetic Microplate Reader (Molecular Devices) with SoftMax® software and used as baseline reading prior to measuring product formation. 100 μl of S-butyrylthiocholine iodide were pipetted into each well with a multiple pipette and mixed. Absorbance at a wavelenght of 405 nm was measured at 1 min, 5 min and 10 min. One unit was defined as the amount of BChE that hydrolyzed 1 micromol of substrate/min.

A specific activity of 720 Units/mg, measured at 25°C with 1 mM butyrylthiocholine in 0.1 M potassium phosphate (pH 8.0), was the standard for purified human BChE. The activity detected using the milk of two negative control mice (0.7 Units/ml, 0.97 mg/ml; 0.84 Unit/ml; 1.16 mg/ml) was subtracted from the activity detected in the milk of the transgenic mice. The results (see Table 3) clearly show that BChE activity was detected in both founder trangenic mice (F0 generation) and in the milk of female offspring (F1 generation).

Analysis of non-denaturing BChE activity gels

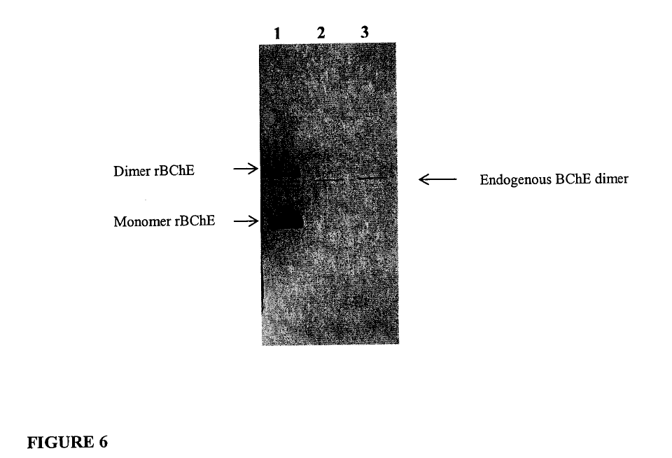

The collected whey phase samples were also electrophoresed on native 4-20% pre-cast TRIS-glycine gels (Invitrogen) at 100 V overnight and 4°C. The gels were then stained for BChE activity with 1 mM of butyrylthiocholine iodide according to the Karnovsky and Roots method (Karnovsky and Roots, Histochem. Cytochem. (1964) 12:219-221). The staining procedure was performed at ambient temperature for two to six hours until the active protein bands were revealed. As can be seen from FIGURE 6, the endogenous mouse BChE present in milk (lanes 2 and 3) migrates at a different size than the recombinant human BChE (lane 1). The recombinant human BChE is produced as a mixture of dimers and monomers, while the endogenous BChE is predominantly a dimer.

The above results demonstrate that recombinant human BChE can be produced and secreted by the mouse mammary gland, with the resultant milk containing levels of up to greater than 1.5 g/L of recombinant human BChE (see Female 4 in Table 3). The secretion of recombinant BChE has no adverse effects on lactation, as shown by the ability of transgenic females to nurse their pups.

Example 3: Production of Recombinant BChE-hSA Fusion Protein in Transgenic Mice

The methods and protocols used for this example, unless otherwise stated, were the same as those used for Example 2.

3.1. Expression construct pBCNN/BChE/hSA

pBCNN/wtBChE/hSA

The vector pBCNN/BChE (see Example 2.1 and FIGURE 4) was digested with XhoI to remove the BChE insert, blunt-ended by filling in with Klenow polymerase in the presence of dNTPs, and CIP treated. Construct pCMV/BChE/hSA (See Example 1.1) was partially digested with NcoI to remove the BChE-hSA encoding sequences, blunt-ended by filling in with Klenow polymerase in the presence of dNTPs, and PmeI digested. The two blunt-ended fragments were ligated to generate pBCNN/wtBChE/hSA. In this construct the signal sequence is the BChE signal sequence.

pBCNN/BChE/hSA

The BstAPI fragment (from 4976 nt to the middle part of BChE) of pBCNN/wtBChE/hSA was replaced with the same BstAPI fragment from pBCNN/BChE (See Example 2.1) to generate pBCNN/BChE/hSA. In this construct the signal sequence is from goat β-casein.

pBCNN/BChE/hSA was digested with NotI, and the resultant NotI-digested linear DNA, free of bacterial sequences, was prepared and used to generate transgenic mice. Briefly, circular expression construct DNA was purified by the cesium chloride gradient technique. This purified DNA was restricted with NotI, electrophoresed, and the linear DNA fragment was gel purified. The DNA fragment was then mixed with cesium chloride and centrifuged at 20°C, 60,000 rpm for 16 to 20 hrs in a Beckman L7 ultracentrifuge using a Ti70.1 rotor (Beckman Instruments, Fullerton, Calif., USA). The DNA band was removed, dialyzed against WFI water for 2-4 hrs, and precipitated in ethanol. The precipitated DNA was resuspended in injection buffer (5 mM Tris pH 7.5, 0.1 mM EDTA, 10 mM NaCl) and dialyzed against the same buffer at 4°C for 8 hrs. Two additional dialysis steps were performed, one for 16 hrs and the second for at least 8 hrs. After dialysis the DNA was quantitated using a fluorometer. Prior to use an aliquot was diluted to 2-3 ng/ml in injection buffer.

3.2 Production of founders and subsequent generations of BChE/hSA transgenic mice.

The production and maintenance of transgenic mice were conducted at McIntyre Transgenic Core Facility of McGill University. Transgenic mice were generated by pronuclear microinjection essentially as described in Hogan et al. , "Manipulating the Mouse Embryo: A Laboratory Manual", Cold Spring Harbor Laboratory (1986. The BCNN/BChE linear fragment was microinjected into 519 fertilized eggs (strain FVB), and 27 pups were born (see Table 2 for details).

At 2-3 weeks of age tail biopsies were taken under anesthesia and DNA was prepared according to standard procedures well known to those skilled in the art, and described in detail, for example, in "Molecular Cloning: Laboratory Manual, 2 ^{nd} Edition", Sambrook et al. , Cold Spring Harbor Laboratory (1989. The presence of the transgene in the genomic DNA was confirmed by PCR analysis as described in sectopm 3.3., Identification of Transgenic Mice , below. Out of 29 tail DNA samples, 1 female founder and one dead pup were confirmed transgene positive.

3.3. Identification of transgenic mice.

The presence of the transgene in mice was confirmed by PCR as described in Example 2.3., except that PCR primer set A was replaced with primer set I, primers ACB712 (5' CTT CCG TGG CCA GAA TGG AT 3') (SEQ ID NO: 11) and ACB884 (5' CCT CAC TCT TGT GTG CAT CG 3') (SEQ ID NO: 20), which amplifies a 462 bp fragment from the 3' end of the transgene spanning the junction of the BChE and albumin sequences.

3.4. Expression of the recombinant BChE-hSA fusion protein in transgenic mice.

Levels BChE activity measured using the Ellman Assay

The Ellman BChE activity assay is performed on the the whey phase of milk collected from the female founder mouse (as described in Example 2.4.). The activity detected using the milk of two negative control mice is subtracted from the activity detected in the milk of the transgenic mouse. This assay will be used to confirm that the recombinant BChE-hSA fusion is catalytically active.

Table 2: Transgenic mice produced via pronuclear microinjection

| | |
|---|---|
| BCNN-BChE construct | |
| Eggs microinjected | 414 |
| Eggs trasferred tp recipients | 265 |
| Recipient mice (average embryos per | 9 (25) |
| recipient) | |
| % Recipient pregnant | 56% |
| Pups born | 28 |
| Pups transgenic (Male/Female; dead; | 6/28 (2/2, 2 dead; 21%) |
| % transgenic) | |
| pBCNN/BChE/hSA | |
| Eggs microinjected | 516 |
| Eggs transferred to recipients | 294 |
| Recipient mice (average embryos per | 13 (26) |
| recipient) | |
| % Recipients pregnant | 61% |
| Pups born | 32 |
| Pups transgenic (Male/Female, dead; | 2/27 (0/1, 1; 7%) |
| % transgenic) | |

Table 3: Transgene copy number and analysis of BChE activity in milk of transgenic mice

| BCNN-BChE | | | | | | |
|---|---|---|---|---|---|---|
| Founder (F0) bred | Copy # | Ellman (mg/L) | F1 transmission | F1 bred | Copy # | Ellman (mg/L) |
| Male A | ˜40 | NA | 14/21 (67%) 6 Males 8 Females | Male 1 | ˜40 | NA |
| Male 2 | ˜40 | NA | | | | |
| Male 3 | ˜40 | NA | | | | |
| Male 4 | ˜40 | NA | | | | |
| Male 5 | ˜40 | NA | | | | |
| Male 6 | ˜40 | NA | | | | |
| Female 1 | ˜40 | 418 | | | | |
| Female 2 | ˜40 | 151 | | | | |
| Female 3 | ˜40 | 388* | | | | |
| Female 4 | ˜40 | 1800 | | | | |
| Female A | ˜10 | 3.5 | ND | ND | ND | ND |
| Female B | ˜40 | 390* | 5/19 (26%) 4 Males 1 Female | Male 7 | ˜40 | NA |
| Male 8 | ˜40 | NA | | | | |
| Male 9 | ND | NA | | | | |
| Male 10 | ND | NA | | | | |
| Female 5 | ND | 910 | | | | |

NA = not applicable.

ND = not done.

* Value represents the average of three independent assays.

Example 4: Production of Recombinant Human BChE in Transgenic Goats

4.1.Hormonal treatment of oocyte donor goats:

Recipient and donor crossbreed goats (mainly Saanen x Nubian) were estrus synchronized by means of an intravaginal sponge impregnated with 60 mg medroxyprogesterone acetate (Veramix®, Pharmacia Animal Health, Ontario, Canada) for 10 days, together with a luteolytic injection of 125 µg clorprostenol (Estrumate®, Schering, Canada) administered intramuscularly 36 hours prior to sponge removal. In addition, for donor goats follicular development was stimulated by a gonadotrophin treatment consisting of 70 mg NIH-FSH-P1 (Folltropin-V ^{®} , Vetrepharm, Canada) and 300 IU eCG (Novormon 5000®, Vetrepharm, Canada) administered intramuscularly 36 h prior to Laparaoscopic Ovum Pick-Up (LOPU).

4.2.Collection of Cumulus Oocyte Complexes (COCs) From donor goats by Laparoscopic Ovum Pick-Up (LOPU).

Cumulus oocyte complexes (COCs) from donor goats were recovered by aspiration of follicle contents (puncture or folliculocentesis) under laparoscopic observation. The laparoscopy equipment used (Richard Wolf, Germany) was composed of a 5 mm telescope, a light cable, a light source, a 5.5 mm trocar for the laparoscope, an atraumatic grasping forceps, and two 3.5 mm "second puncture" trocars. The follicle puncture set was composed of a puncture pipette, tubing, a collection tube, and a vacuum pump. The aspiration pipette was made using an acrylic pipette (3.2 mm external diameter, 1.6 mm internal diameter), and a 20G short bevel hypodermic needle, which was cut to a length of 5 mm and fixed into the tip of the pipette with instant glue. The connection tubing was made of clear plastic tubing with an internal diameter of 5 mm, and connected the puncture pipette to the collection tube. The collection tube was a 50 ml centrifuge tube with an inlet and an outlet available in the cap. The inlet was connected to the aspiration pipette, and the outlet was connected to a vacuum line. Vacuum was provided by a vacuum pump connected to the collection tube by means of clear plastic 8 mm tubing. The vacuum pressure was regulated with a flow valve and measured as drops of collection medium per minute entering the collection tube. The vacuum pressure was typically adjusted to 50 to 70 drops per minute.

The complete puncture set was washed and rinsed 10 times with tissue culture quality distilled water before gas sterilization, and one time before use with collection medium, M199 + 25 mM HEPES (Gibco) supplemented with penicillin, streptomycin, kanamycin, bovine serum albumin, and heparin). Approximately 0.5 ml of this medium was added to the collection tube to receive the oocytes.

Donors were deprived of food for 24 hours and of water for 12 hours prior to surgery. The animals were pre-anesthetized by injection of diazepam (0.35 mg/kg body weight) and ketamine (5 mg/kg body weight). Thereafter, anesthesia was maintained by administration of isofluorane via endotrachial intubation. Preventive antibiotics ( e.g. , oxytetracycline) and analgesic/anti-inflammatories ( e.g. , flunixine) were administered by intramuscular injection in the hind limbs. The surgical site was prepared by shaving the abdominal area, then scrubbing first with soap and water and then with a Hibitaine:water solution, followed by application of iodine solution.

A small incision/puncture was made with a scalpel blade about 2 cm cranial from the udder and about 2 cm left from the midline. The 5 mm trocar was inserted and the abdominal cavity was inflated with filtered air through the trocar sleeve gas valve. The laparoscope was inserted into the trocar sleeve. A second incision was made about 2 cm cranial from the udder and about 2 cm right from the midline, into which was inserted a 3.5 mm trocar. The trocar was removed, and the forceps was inserted. A third incision was made about 6 cm cranial to the udder and about 2 cm right from the midline. The second 3.5 mm trocar and trocar sleeve was inserted into this incision. The trocar was removed and the aspiration pipette connected to the vacuum pump and the collection tube was inserted therein.

After locating the reproductive tract below the bladder, the ovary was exposed by pulling the fimbria in different directions, and the number of follicles available for aspiration was determined. Generally, follicles greater than 2 cm were considered eligible for aspiration. The follicles were punctured one by one and the contents aspirated into the collection tube under vacuum. The needle was inserted into the follicle and rotated gently to ensure that as much of the follicle contents as possible were aspirated. After >10 follicles were aspirated and/or before moving to the other ovary, the pipette and tubing were rinsed using collection media from a sterile tube.

4.3.In vitro maturation of oocytes collected by LOPU

To each collection tube containing cumulus oocyte complexes (COCs) was added about 10 ml of searching medium, EmCare® supplemented with 1% heat inactivated Fetal Bovine Serum (FBS). The resulting solution was aspirated into a grid search plate and transferred to Petri dishes containing the same medium for the purpose of scoring each COC for amount and expansion of cumulus. The COCs were then washed with i n vitro maturation (IVM) medium; (M199 + 25 mM HEPES supplemented with bLH, bFSH, estradiol β-17, pyruvate, kanamycin and heat-inactivated EGS) that had been equilibrated in an incubator under 5% CO ₂ at 35.5°C for at least 2 hours. The COCs were pooled in groups of 15-25 per droplet of IVM medium, overlayed with mineral oil, and incubated in 5% CO ₂ at 35.5°C for 26 hours.

4.4.Preparation of semen for in vitro fertilization

Fresh semen was collected from 2 adult Saanen males of known fertility. After collection, sperm capacitation was achieved as follows. A 5 µl aliquot of fresh semen was diluted in 500 µl warm modified Defined Medium (mDM) comprising NaCl, KCl, NaH ₂ PO ₄ ·H ₂ O, MgCl ₂ ·6H ₂ O, CaCl ₂ ·2H ₂ O, glucose, 0.5% phenol red, Na-Pyruvate, NaHCO ₃ , gentamicin, and BSA. The solution was allowed to stand at room temperature in the absence of light for 3 hours. An additional 1 ml of mDM solution was added and 100 µl of the resulting solution was overlaid on a 45%:90% Percoll ^{®} gradient [Percoll ^{®} (Sigma P1644) in modified Sperm Tyrodes Lactate (SPTL) solution] in a conical centrifuge tube. The solution was centrifuged on the Percoll gradient at 857 x g for 30 minutes. The pellet was resuspended in mDM solution and centrifuged at the same speed for 10 minutes. The pellet was re-suspended in capacitation medium (mDM, supplemented with 8b-cAMP, Ionomycin, and Heparin). The resuspended semen was cultured at 38.5°C under 5% CO ₂ for 15 minutes. The sperm concentration was then adjusted to final concentration of 20 x 10 ⁶ sperm/ml by addition of mDM solution.

4.5In vitro fertilization of oocytes

The expanded cumulus cells were partially removed from the matured COCs by pipetting repeatedly through two fine-bore glass pipettes (200 and 250 µm internal diameter), leaving one layer of cumulus cells on the zona. The oocytes were washed with in vitro fertilization (IVF) medium, a modified Tyrode's albumin lactate pyruvate (TALP), and transferred to 40 µl droplets of the same medium (15-20 oocytes per 40 µl droplet) under mineral oil. A 5 µl aliquot of the capacitated sperm suspension (20 x 10 ⁶ sperm/ml), prepared as described in Example 4.4., was added to each 40 µl droplet. The inseminated oocytes were cultured at 38.5 C in 5% CO ₂ for 15-16 hours.

4.6Pronuclear microinjection of oocytes

After culturing for 15-16 hours, the cumulus cells were stripped from the inseminated oocytes (zygotes) by repeated pipetting as described above. The zygotes were then observed for pronuclear formation using an Olympus stereomicroscope. To improve pronucleus visualization, the zygotes were washed in EmCare® (PETS, cat. # ECFS-100) supplemented with 1% Fetal Bovine Serum (FBS), (Gibco BRL, Australian or New Zealand sourced, heat inactivated at 56 °C for 30 minutes), then centrifuged at 10,400 x g for 3 minutes before observation. Zygotes with visible pronuclei were selected for microinjection and transferred to 50 µl droplets of temporary culture medium (INRA Menezo B2, Meditech cat. #CH-B 04001 supplemented with 2.5% FBS) during manipulation. The zygotes were then transferred to 50 µl droplets of EmCare® + 1% FBS (about 20 zygotes per droplet) and microinjected with the BCNN/BChE linear fragment from Example 2.1. (3 ng/ml of the DNA in a buffer of 5m M Tris, 0.1 mM EDTA. 10m M NaCl buffer, pH 7.5). The injected zygotes were washed and cultured in temporary culture medium to await transfer to recipients.

4.7Transfer of embryos to oviduct of recipient goats and birth of kids

Adult goats of various breeds including the Boer, Saanen, and Nubian breeds were used as recipients. They were estrus synchronized by means of an intravaginal sponge impregnated with 60 mg medroxyprogesterone acetate (Veramix®, Pharmacia Animal Health, Ontario, Canada) left in place for 9 days, together with a luteolytic injection of 125 µg clorprostenol (Estrumate®, Schering, Canada) and 500 IU eCG (Novormon 5000®, Vetrepharm, Canada) administered intramuscularly 36 hours prior to sponge removal . Sponges were inserted into the recipient goats on the same day as the donor goats but removed approximately 15 hours earlier. Each recipient was subsequently treated with an intramuscular injection of 100 µg GnRH (Factrel ®, 2.0 ml of 50 µg/ml solution), 36 hours after sponge removal. The recipients were tested for estrus with a vasectomized buck at 12 hour intervals beginning 24 hours after sponge removal and ending 60-72 hours after sponge removal.

Recipient goats were fasted, anesthetized, and prepared for surgery following the same procedures previously described for donor goats. They also received preventive antibiotic therapy and analgesic/anti-inflammatory therapy, as described for donors. Prior to surgery, a laparoscopic exploration of each eligible recipient was performed to confirm that the recipient had one or more recent ovulations (as determined by the presence of corpora lutea on the ovary), and a normal oviduct and uterus. The laparoscopic exploration was carried out to avoid performing a laparotomy on an animal which had not responded properly to the hormonal synchronization protocol described above. Two incisions were made (one 2 cm cranial to the udder and 2 cm left of the midline, and the other 2 cm cranial to the udder and 2 cm right of the midline) and the laparoscope and forceps were inserted as described above. The ovaries were exposed by pulling up the fimbria with the forceps, and the number of ovulations present as well as the number of follicles larger than about 5 mm diameter was noted. Recipients with at least one ovulation present and having a normal uterus and oviduct were eligible for transfer. A mid-ventral laparotomy incision of approximately 10 cm length was established in eligible recipients, the reproductive tract was exteriorized, and the embryos were implanted into the oviduct ipsilateral to the ovulation(s) by means of a TomCat® catheter threaded into the oviduct from the fimbria. The incisions were closed and the animal was allowed to recover in a post-op room for 3 days before being returned to the pens. Skin sutures were removed 7-10 days after surgery.

Recipients were scanned by transrectal ultrasonography using a 7.5 Mhz linear array probe to diagnose pregnancy at 28 and 60 days after transfer.

Newborn kids were removed from does at birth to prevent disease transmission from doe to kid by ingestion of doe's raw colostrum and/or milk, exposure to doe's fecal matter or other potential sources of disease. Kids were fed thermorized colostrum for the first 48 hours of life, and pasteurized doe milk thereafter until weaning.

4.8. Identification of transgenic goats

Blood and tissue samples were taken from putative transgenic kids at approximately 4 days after birth, and again at approximately two weeks after birth. At each sampling interval, about 2-7 ml blood sample was collected from each kid into an EDTA vacutainer, and stored at 4°C for up to 24 hours until use. Tissue samples were obtained by clipping the ear tip of each kid, and stored at 20°C until use. Genomic DNA was isolated from the blood samples using a QIAamp ^{®} DNA Blood Mini Kit (Qiagen, Cat. # 51106), and from the tissue samples using DNeasy ^{®} Tissue Kit (Qiagen, cat #69506). For each sample, the DNA was eluted in 150-200 µl 0.1x buffer AE and stored at 4°C until ready to use.

PCR screening was performed on each DNA sample to determine the presence of the BChE-encoding transgene. Genomic DNA samples were diluted using nuclease-free water to a concentration of 5 ng/µl. A 20 µl portion of the diluted DNA was added to a 0.2 ml Ready-To-Go™ PCR tube containing a PCR bead, together with 5 µl 5 x primer mix containing dUPT (Amersham Bioscience, cat. #272040) and UDG (Invitrogen, cat. #18054-015). The primer sets used were identical to the ones used in the PCR analysis of Example 2.3., except for primer set C. In this case, primer set C was replaced with the primers Acb256 (5' GAG GAA CAA CAG CAA ACA GAG 3') (SEQ ID NO: 21) and Acb312 (5' ACC CTA CTG TCT TTC ATC AGC 3') (SEQ ID NO: 22), which amplify a 360 bp portion of the endogenous goat β-casein gene. This primer set serves as in internal positive control to indicate that the extracted DNA can be amplified by PCR.

The sample was subjected to thermal cycling and then applied to a 1% agarose gel. Negative controls (genomic DNA isolated from non-transgenic animals) and positive controls (genomic DNA from non-transgenic animals spiked with the microinjected BCNN/BChE linear fragment) were also included. Samples which exhibited a band corresponding to the positive control were deemed positive. Based on this PCR analysis, a total of 6 transgenic goats were identified (5 females and 1 male).

The presence of the transgene was confirmed by Southern blotting as described in Example 2.3. The expected size bands were detected for all transgenic founders (F0 generation), and transgene copy number was estimated to be between about 4-50 copies (see Table 5). Fluorescent in situ hybridization (FISH) was performed as described in Keefer et al. , Biol. Reprod. (2001) 64:849-856 in order to determine the number of chromosomal integration sites (Table 5).

Table 4: Transgenic goats produced via nuclear proinjection

| | |
|---|---|
| Donor goats aspirated | 68 |
| Follicles aspirated (ave. per donor goat) | 1410 (20.7) |
| Oocytes recovered (ave. per donor goat, recovery rate) | 1256 (18.5, 89%) |
| Zygotes microinjected (% of oocytes recovered) | 724 (58%) |
| Zygotes transferred (% of microinjected) | 635 (88%) |
| Recipient goats (ave. embryos per recipient) | 92 (6.9) |
| Recipient pregnant at 28 days (% pregnant) | 48 (52%) |
| Kids born (ave. per recipient) | 61 (1.7) |
| Kids transgenic (Male/Female; % of kids born) | 6 (5/1; 10%) |

Table 5: Transgene copy number and chromosomal integration sites of founder transgenic goats.

| Founder goat | Trangene | Integration sites |
|---|---|---|
| (F0 generation) | copy number | (by FISH) |
| Male 1 | ˜5-10 | 3 |
| Female 1 | ˜2-5 | 2 |
| Female 2 | ˜2-5 | 2-3 |
| Female 3 | ˜20 | 1-2 |
| Female 4 | ˜5-10 | 2-3 |
| Female 5 | ND | 1 |

ND = not done

4.9.Induction of lactation

Female founders were induced to lactate at 3-4 months of age in order to confirm the expression of recombinant BChE in milk. For such purpose they were hormonally stimulated with estradiol cypionate (0.25 mg/KBW) and Progesterone (0.75 mg/KBW) every 48 hrs for two weeks, followed by treatment with dexamethasone (8 mg/ goat /day) for 3 days. In general, milk production started during the dexamethasone treatment and the animals were milked twice per day for as long as necessary to produce enough material for further testing.

4.10. Analysis of BChE-activity in the milk of transgenic goats

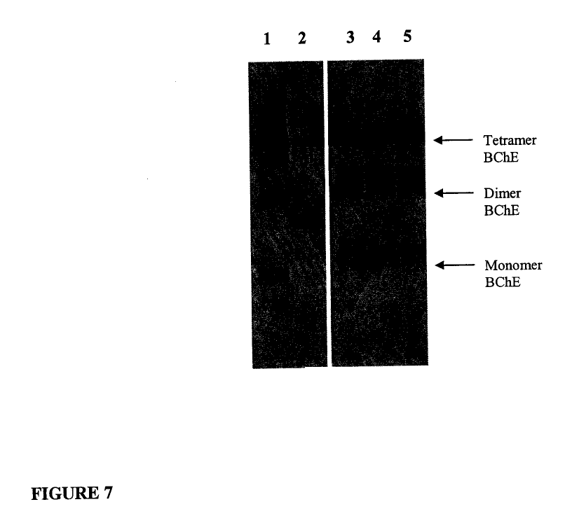

The presence and activity of recombinant BChE in the milk of transgenic goats was analyzed by non-denaturing BChE-activity gel as described in Example 2.4. Such analysis (see FIGURE 7) showed that active recombinant BChE is produced in the milk of transgenic goats. The recombinant BChE is present in both a tetramer and dimer form, and to a lesser extent in the monomer form.

4.11. Purification of recombinant BChE from the milk of transgenic goats

Clarification of milk

20 ml of milk containing recombinant BchE was diluted to 60 ml with 20 mM phosphate buffer (pH 7.4). Ammonium sulfate (15 grams) was slowly added to the diluted milk, and the mixture was agitated until all ammonium sulfate solids were dissolved. This liquid was incubated at 4°C for one hour, and then phase separated by centrifugation at 20,000 x g for 30 min. The liquid phase containing recombinant BChE was harvested and then dialyzed overnight against 20 mM phosphate buffer (pH 7.4), 100 mM sodium chloride, and 1 mM EDTA. 75 ml of liquid containing recombinant BChE was recovered and further clarified by filtration using a 0.2 µm filter. The recovery of BchE based on activity (Ellman reaction) was 50%.

Affinity chromatography with Procainamide

An affinity resin was prepared using standard protocols with Procainamide (Sigma) and Activated CH Sepharose (Amersham). A column was packed with 20 ml Procainamide affinity resin and equilibrated with 20 mM phosphate buffer (pH 7.4), 100 mM sodium chloride, and 1 mM EDTA. The 75ml of liquid containing recombinant BChE was loaded onto the column at a linear flow rate of 50 cm/hr. The column was washed with 20 mM phosphate buffer (pH7.4), 150 mM sodium chloride, and 1 mM EDTA. BChE was eluted with 20 mM phosphate buffer (pH 7.4), 500 mM sodium chloride, and 1 mM EDTA. The eluent containing recombinant BChE was dialysed against 20 mM phosphate buffer (pH 7.4), 50 mM sodium chloride, and 1 mM EDTA. A total of 50 ml of liquid containing recombinant BChE was recovered after dialysis. The recovery of BchE after this step was 90%.

Anion exchange chromatography

A column was packed with 20 ml HQ50 resin (Applied Biosystems) and equilibrated with 20 mM phosphate buffer (pH 7.4), 50 mM sodium chloride, and 1 mM EDTA. The 50 ml of liquid containing recombinant BChE was recovered after affiinity chromatography was loaded onto the column at a linear flow rate of 100 cm/h. The column was washed with 20 mM phosphate buffer (pH 7.4), 50 mM sodium chloride, and 1mM EDTA. Purified recombinant BChE was eluted with 20 mM phosphate buffer (pH7.4), 250 mM sodium chloride, and 1mM EDTA. This eluent was dialyzed against 20 mM phosphate buffer (pH 7.4), 100 mM sodium chloride, and 1 mM EDTA, and then further concentrated to a final purfied concentration of 15 mg/ml of protein. The recovery of BChE after this step was 90%.

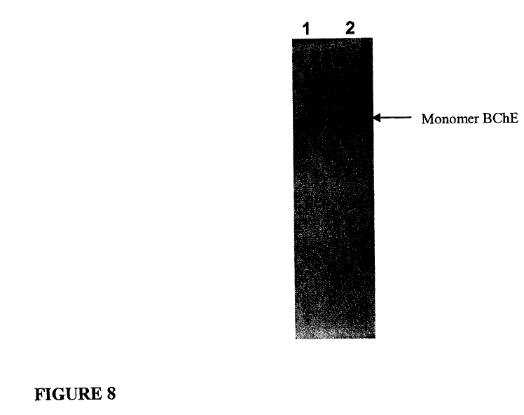

In order to estimate the purity of the purified recombinant BChE, a 0.2 µg sample was subjected to denaturing SDS-PAGE electrophoresis under reducing conditions. The gel was then silver stained to show total protein of the sample (see FIGURE 8). Note that all of the purified recombinant BChE migrates as a monomer on this gel, due to reduction of the protein samples with beta-mercaptoethanol prior to loading on the gel, and to denaturation of the proteins during electrophoresis. This analysis was used to estimate that the purified recombinant BchE is >80% pure (compare band intensity of the 0.2 µg sample versus that of 0.2 µg of the positive control).

Example 5: Production of Recombinant BChE-hSA Fusion Protein in Transgenic Goats

Trangenic goats expressing a recombinant BChE-hSA fusion protein may be generated by nuclear transfer. The nuclear donors are primary fetal goat cells stably transfected with the BCNN/BChE/hSA linear fragment (from Example 3.1.).

5.1. Generation of stably transfected cell lines

Primary fetal goat cells were derived from day 28 kinder fetuses recovered from a pregnant Saanen breed female goat, and cultured for 3 days prior to being cryopreserved. Chromosome number (2n=60) and sex analysis was performed prior to use of cells for transfection experiments. Under the culture conditions used, all primary lines had a normal chromosome count indicating the absence of gross chromosomal instability during culture.

Transfections were performed as described in Keefer et al. , Biol. Reprod. (2001) 64:849-856, with the following modifications: Female primary lines were thawed and at passage 2, co-transfected with the linearized BCNN/BChE/hSA fragment and the linearized pSV40/Neo selectable marker construct (Invitrogen). The pSV40/Neo linear fragment was generated by restriction of the vector with XbaI and NheI, followed by purification of the fragment as described in Example 2.1. Stably transfected cell lines were selected with G418 and frozen by day 21 (day 0 = transfection date).

Four stably transfected cell lines have been derived by this procedure. In all cases the presence of the transgene has been confirmed by Southern Analysis and by Fluorescence In Situ Hybridization (FISH). Transfected cell lines for which integration of the transgene is confirmed will serve as donors for nuclear transfer.

5.2. Oocyte donor and recipient goats

Intravaginal sponges containing 60 mg of medroxyprogesterone acetate (Veramix ^{®} ) are inserted into the vagina of donor goats (Alpine, Saanen, and Boer cross bred goats) and left in place for 10 days. An injection of 125 μg cloprostenol is given 36 hrs before sponge removal. Priming of the ovaries is achieved by the use of gonadotrophin preparations, including FSH and eCG. One dose equivalent to 70 mg NIH-FSH-P1 of Ovagen™ is given together with 400 IU of eCG (Equinex) 36h before LOPU (Laparoscopic Oocyte Pick-Up).

Recipients are synchronized using intravaginal sponges as described above for donor animals. Sponges are removed on day 10 and an injection of 400 IU of eCG is given. Estrus is observed 24-48 hrs after sponge removal and embryos are transferred 65-70 hrs after sponge removal.

5.3. Laparoscopic oocyte Pick-Up (LOPU) and embryo transfer

These procedures are performed essentially as described in Examples 4.2. and 4.7.

Donor goats are fasted 24 hours prior to laparoscopy. Anesthesia is induced with intravenous administration of diazepam (0.35 mg/kg body weight) and ketamine (5 mg/kg body weight), and is maintained with isofluorane via endotrachial intubation. Cumulus-oocyte-complexes (COCs) are recovered by aspiration of follicular contents under laparoscopic observation.

Recipient goats are fasted and an aesthetized in the same manner as the donors. A laparoscopic exploration is performed to confirm if the recipient has had one or more recent ovulations or corpora lutea present on the ovaries. An average of 11 nuclear transfer-derived embryos (1-cell to 4-cell stage) are transferred by means of a TomCat® catheter threaded into the oviduct ipsilateral to ovulation(s). Donors and recipients are monitored following surgical procedures and antibiotics and analgesics are administered according to approved procedures.

5.4. Oocyte maturation

COCs are cultured in 50 μl drops of maturation medium covered with an overlay of mineral oil and incubated at 38.5-39°C in 5% CO2. The maturation medium consists of M199H (GIBCO) supplemented with bLH, bFSH, estradiol β-17, sodium pyruvate, kanamycin, cysteamine, and heat inactivated goat serum. After 23 to 24 hrs of maturation, the cumulus cells are removed from the matured oocytes by vortexing the COCs for 1-2 min in EmCare® containing hyaluronidase. The denuded oocytes are washed in handling medium (EmCare® supplemented with BSA) and returned to maturation medium. The enucleation process is initiated within 2 hr of oocyte denuding. Prior to enucleation, the oocytes are incubated in Hoechst 33342 handling medium for 20-30 minutes at 30-33°C in air atmosphere.

5.5. Nuclear transfer

Oocytes are placed into manipulation drops (EmCare® supplemented with FBS) covered with an overlay of mineral oil. Oocytes stained with Hoechst are enucleated during a brief exposure of the cytoplasm to UV light (Zeiss Filter Set 01) to determine the location of the chromosomes. Stage of nuclear maturation is observed and recorded during the enucleation process.

The enucleated oocytes and dispersed donor cells are manipulated in handling medium. Transgenic donor cells are obtained following either in vitro transfection (see Example 5.1.) or biopsy of a transgenic goat. Donor cells are prepared by serum starving for 4 days at confluency. Subsequently they are trypsinized, rinsed once, and resuspended in Emcare® with serum. Small (<20 μm) donor cells with smooth plasma membranes are picked up with a manipulation pipette and slipped into perivitelline space of the enucleated oocyte. Cell-cytoplast couplets are fused immediately after cell transfer. Couplets are manually aligned between the electrodes of a 500 μm gap fusion chamber (BTX, San Diego, CA) overlaid with sorbitol fusion medium. A brief fusion pulse is administered by a BTX Electrocell Manipulator 200. After the couplets have been exposed to the fusion pulse, they are placed into 25 μl drops of medium overlaid with mineral oil. Fused couplets are incubated at 38.5-39°C. After 1 hr, couplets are observed for fusion. Couplets that have not fused are administered a second fusion pulse.

5.6. Oocyte activation and culture

Two to three hours after application of the first fusion pulse, the fused couplets are activated using calcium ionomycin and 6-dimethylaminopurine (DMAP) or using calcium ionomycin and cycloheximide/cytochalasin B treatment. Briefly, couplets are incubated for 5 minutes in EmCare® containing calcium ionomycin, and then for 5 minutes in EmCare® containing BSA. The activated couplets are cultured for 2.5 to 4 hrs in DMAP, then washed in handling medium and placed into culture drops (25 μl in volume) consisting of G1 medium supplemented with BSA under an oil overlay. Alternately, following calcium ionomycin treatment, the activated couplets are cultured for 5 hrs in cycloheximide and cytochalasin B, washed, and placed into culture. Embryos are cultured 12 to 18 hrs until embryo transfer. Nuclear transfer derived embryos are transfered on Day 1 (Day 0 = day of fusion) into synchronized recipients on Day 1 of their cycle (Day 0 = estrus).

5.7. Identification of stably transfected cell lines and of transgenic goats

Following selection of transfected cell lines, genomic DNA is isolated from cell pellets using the DNeasy ^{®} Tissue Kit (Qiagen, cat #69506). For each sample, the DNA is eluted in 150-200µl 0.1X AE buffer and stored at 4°C until ready to use.

For confirmation of the presence of the transgene in nuclear transfer derived offspring, genomic DNA is extracted from the blood and ear biopsy of 2 week old kids using standard molecular biology techniques. The genomic DNA is isolated from the blood samples using a QIAamp ^{®} DNA Blood Mini Kit (Qiagen, Cat. # 51106), and from the tissue samples using DNeasy ^{®} Tissue Kit (Qiagen, cat #69506). For each sample, the DNA is eluted in 150-200 µl 0.1X AE buffer and stored at 4°C until use.

The presence of the transgene, in stably transfected cells and in transgenic goats, is confirmed by PCR as described in Example 2.3., except for the following modifications. PCR primer set A is replaced with primer set I: Primers ACB712 (5' CTT CCG TGG CCA GAA TGG AT 3') (SEQ ID NO: 11) and ACB884 (5' CCT CAC TCT TGT GTG CAT CG 3') (SEQ ID NO: 20) which amplify a 462 bp fragment from the 3' end of the transgene spanning the junction of the BChE and albumin sequences. Primer set C is replaced with the primers Acb256 (5' GAG GAA CAA CAG CAA ACA GAG 3') (SEQ ID NO: 21) and Acb312 (5' ACC CTA CTG TCT TTC ATC AGC 3') (SEQ ID NO: 22), which amplify a 360 bp portion of the endogenous goat β-casein gene. This primer set serves as in internal positive control to indicate that the extracted DNA can be amplified by PCR.

The presence of the transgene, in stably transfected cells and in transgenic goats, is also confirmed by Southern blotting as described in Example 2.3. Fluorescent in situ hybridization (FISH) is performed as described in Keefer et al. , Biol. Reprod. (2001) 64:849-856 in order to determine the number of chromosomal integration sites. The FISH probe contains only sequences from the insulator region of the transgene.

Example 6: Pharmacokinetic Studies of Recombinant BChE Produced by Transgenic Mammals

Residence time of recombinant BChE in the circulation of guinea pigs is determined as described by Raveh et al. , Biochemical Pharmacology (1993) 42:2465-2474. A sample BchE enzyme, isolated from the milk of transgenic mammal, is dialyzed against sterile phosphate-buffered saline, pH 7.4. The dialyzed enzyme (50-500 units in a volume of ~250 µl) is administered intravenously into the tail vein of guinea pigs. The injection doses are chosen to be sufficient to provide a plasma concentration of recombinant BChE well above the level of endogenous BChE, as estimated by the Ellman assay. At various time intervals, heparinized blood samples (5-10 ul) are withdrawn from the retro-orbital sinus or the toe of the animals and diluted 15 to 20-fold in distilled water at 4°C. The BchE activity in the blood sample is determined using butyrylthiocholine as the substrate for BChE using the assay of Ellman et al. , (1961). Endogenous ChE activity is subtracted from the result. The clearance of recombinant BchE from the circulation is calculated over time.

To test the efficacy of recombinant BChE in prevention of organophosphate poisoning, nerve agents (soman, VX, sarin or GF) are administered intravenously into the tail vein of guinea pigs in a volume of 100 ul PBS. Animals are observed for 24 hours, and the degree of organophosphate poisoning symptomology recorded. Specifically, percent survival is calculated. Blood samples are also taken at 10-20 min post nerve agent injection and assayed for residual BchE activity. The level of BChE activity following administration of a nerve agent is a measure of the potency of the recombinant BChE.

Example 7: BChE Expression Constructs Based on the WAP Promoter

7.1. Introduction

Whey acidic protein (WAP), the major whey protein in mammals, is expressed at high levels exclusively in the mammary gland during late pregnancy and lactation. The genomic locus of the murine WAP gene consists of 4.4 kb of 5' flanking promoter sequence, 2.6 kb of coding genomic sequence, and 1.6 kb of 3' flanking genomic DNA. The WAP promoter may be used to drive expression of heterologous proteins in the mammary gland of transgenic mammals (Velander et al. , Proc. Natl. Acad. Sci. USA (1992) 89:12003-12007).

An expression construct based on the whey acidic protein (WAP) promoter, can be used to preferentially express BChE in milk of transgenic animals. In one embodiment, the construct is assembled by inserting a BChE-encoding sequence between the WAP promoter (position -949 to +33 nt) at the 5' end, and the WAP coding genomic sequence (843 bp; the last 30 base of Exon 3, all of intron 3, and exon 4 including 70 bp of 3' UTR) at the 3' end. The expression construct also includes two copies of an insulator element from the chicken globin locus. The BChE-encoding sequence may contain the BChE signal sequence or the WAP signal sequence. The BChE-encoding sequence may also contain an epitope tag ( e.g. , myc and/or his).

In one embodiment, the contruct comprises the WAP gene promoter, the WAP signal sequence, a BChE-encoding sequence, and the coding and 3' genomic sequences of the WAP gene. This WAP signal sequence is added using a nucleic acid sequence encoding part of the 5' untranslated region and the 19 amino acid signal peptide of the murine WAP gene (position -949 to +89, Hennighausen et al. , Nucl. Acids Res. (1982) 10:3733-3744). The BChE encoding fragment is generated by PCR of a BChE cDNA ( e.g. , ATCC #65726) using a 5' primer containing the 90 bp sequence signal sequence flanked by a KpnI restriction endonuclease recognition site, and 3' primers containing a KpnI restriction endonuclease recognition site and 3' BChE cDNA sequences. The amplification is performed to maintain the correct reading frame. This PCR product is then inserted at the KpnI site at the first exon of WAP. The vector is prepared for microinjection or transfection by digestion with NotI restriction endonuclease and purification of the linear fragment.

7.2. Generation of the expression construct pWAP/BChE

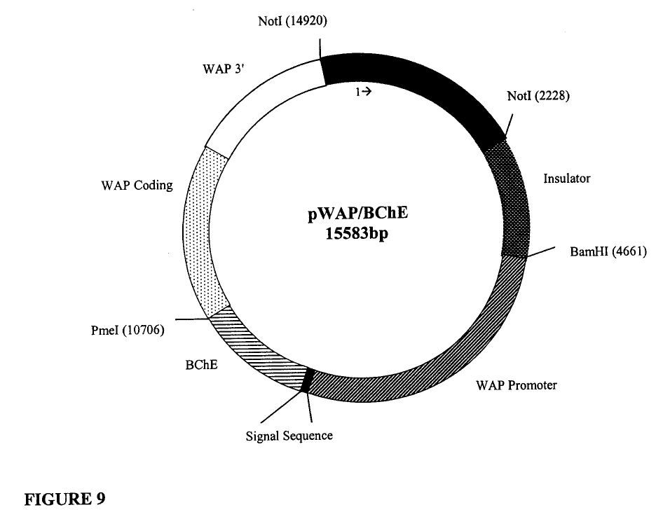

The expression contruct pWAP/BChE (see FIGURE 9) may be prepared as follows:

Step 1: PCR amplification of WAP 3' genomic sequences

The WAP 3' genomic sequence is PCR amplified from mouse genomic DNA with the following primers: WAP-p1 (5' AAT TGG TAC CAG CGG CCG CTC TAG AGG AAC TGA AGC AGA GAC CAT GC 3') (SEQ ID NO: 23) and WAP-p2 (5' GCT GCT CGA GCT TGA TGT TTA AAC TGA TAA CCC TTC AGT GAG CAG CCG ATA TAT GTT TAA ACA TGC GTT GCC TCA TCA GCC TTG TTC 3') (SEQ ID NO: 24). The PCR product is then restricted with XhoI and NotI.

Step 2: PCR amplification of WAP coding genomic sequences

The WAP coding genomic sequence (2630 bp) is PCR amplified from mouse DNA with the primers WAP-p3 (5' ATA TAT GTT TAA ACA TGC GTT GCC TCA TCA GCC TTG TTC 3') (SEQ ID NO: 25) and WAP-p4 (5' ATG TTC TCT CTG GAT CCA GGA GTG AAG G 3') (SEQ ID NO: 26). The PCR product is then restricted with PmeI and BamHI.

Step 3: PCR amplification of the BChE encoding sequence

The BChE encoding sequence (2370 bp) is PCR amplified from a pBChE cDNA with the primers: BChE-p1 (5' ATT TCC CCG AAG TAT TAC 3') (SEQ ID NO: 27) and BChE-p2 (5' TGA TTT TCT GTG GTT ATT 3') (SEQ ID NO: 28). The PCR product is then blunt ended.

Step 4: Ligation of the WAP coding and 3' genomic sequences with the BChE encoding sequence

The pBluescript vector is restricted with KpnI and Sac II. A linker formed by annealing of the primer sequences Linker-p1 (5' GGA CCG GTG TTA ACG ATA TCT CTA GAG CGG CCG CT 3') (SEQ ID NO: 29) and Linker-p2 (5' CCG GAG CGG CCG CTC TAG AGA TAT CGT TAA CAC CGG TCC GC 3') (SEQ ID NO: 30) is inserted to generate additional restriction enzyme sites (KpnI, NotI, XbaI, EcoRV, HpaI, AgeI and SacII). The new vector is recircularized and then then restricted with EcoRV. The BChE encoding PCR product of Step 3 is then blunt-ended, and ligated to this vector.

This new construct is restricted with XhoI and NotI, and the WAP 3' genomic sequence PCR product from Step 1 is inserted. This construct is then restricted with PmeI and BamHI and the 2.6 kb WAP coding genomic sequence PCR product of Step 2 is inserted, to generate a construct wherein the BChE-encoding sequence was linked at its 3' end to the WAP coding and 3' genomic sequences.

Step 5: PCR amplification of the chicken β-globin insulator sequence

The insulator fragment is derived from PCR amplification of chicken genomic DNA with the primers Insulator-p1 (5' TTT TGC GGC CGC TCT AGA CTC GAG GGG ACA GCC CCC CCC CAA AG 3') (SEQ ID NO: 31) and Insulator-p2 (5' TTT TGG ATC CGT CGA CGC CCC ATC CTC ACT GAC TCC GTC CTG GAG TTG 3') (SEQ ID NO: 32). The PCR product is restricted in two independent reactions; one with NotI and XhoI, and one with BamHI and SalI. The two restricted fragments are then ligated together to generate a 2 kb dimerized insulator fragment with NotI and BamHI sites on either end.

Step 6: Ligation of the WAP promoter sequence with the insulator fragment

A pBluescript clone containing the 4.4 kb WAP promoter in the pBluescript plasmid (clone 483, described in Velander et al. , Proc. Natl. Acad. Sci. USA (1992) 89:12003-12007) is restricted with SacII and NotI. A linker formed by annealing of the primer sequences Linker-p3 (5' GGA CTA GTT GAT CAG CGG CCG CTA TAG GAT CC 3') (SEQ ID NO: 33) and Linker-p4 (5' GGC CTG GAT CCT ATA GCG GCC GCT GAT CAA CTA GTC CGC 3') (SEQ ID NO: 34) is inserted to generate a recircularized construct of the 4.4kb WAP promoter containing additional restriction sites (SacII, SpeI, BclI, NotI and BamHI). This new construct is then restricted with Not I and BamHI and ligated to the insulator fragment from Step 5.

Step 7: Generation of pWAP/BChE

The BChE/WAP coding and 3' genomic sequence construct from Step 4 is then restricted with SacII and AgeI. The 6.8 kb fragment containing the insulator and WAP promoter is isolated from the construct of Step 6 by restriction with SacII and AgeI. These two fragments are ligated to form pWAP/BChE. This final construct contains the dimerized chicken β-globin gene insulator followed by the WAP 4.4 kb promoter, the BChE gene, and the WAP 2.6 kb coding and 1.6 kb 3' genomic sequences (See FIGURE 9).

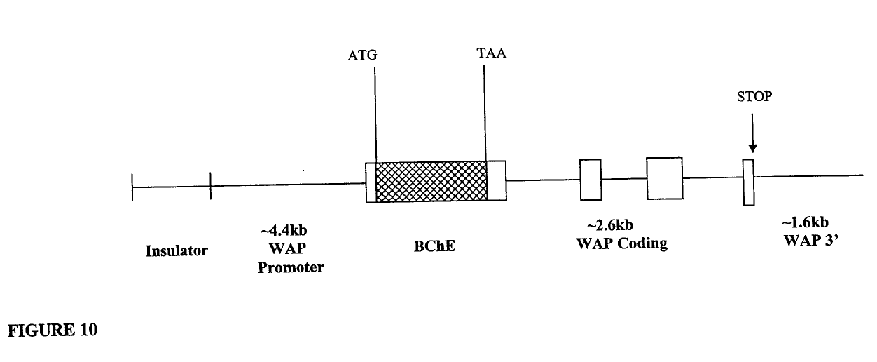

For microinjection or transfection, pWAP/BChE is linearized by NotI digestion to remove the vector sequences. This linearized fragment contains the dimerized insulator, the WAP promoter and signal sequence, the BChE-encoding sequence, and WAP coding and 3' genomic regions (See FIGURE 10).

Example 8: Expression Constructs for the Production of Recombinant BChE

in the Urine of Transgenic Mammals

8.1.Uromodulin promoter

Uromodulin, a 90 kD glycoprotein secreted from the epithelial cells of the thick ascending limbs and the early distal convoluted tubule in the kidney, is the most abundant protein in urine and is evolutionarily conserved in mammals (Badgett and Kumar, Urologia Internationalis (1998) 61:72-75). Thus, the uromodulin promoter is a good candidate for driving the production of recombinant proteins in cells of the kidney, which will then secrete said proteins into the urine.

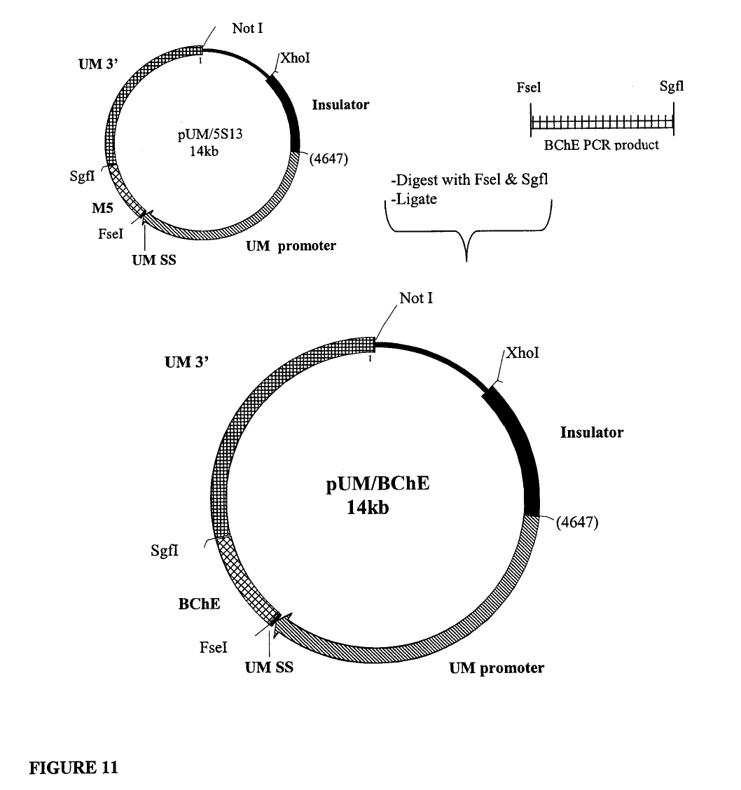

An expression construct comprising a uromodulin promoter and encoding a spider silk protein, pUM/5S13, may be used for the construction of a new expression construct, pUM/BChE, in which the expression of a BChE encoding sequence is controlled by the uromodulin promoter (See FIGURE 11). The parent pUM/5S13 expression construct contains, in this order:

A 2.4 kb fragment of the chicken β-globin insulator;

A 3.4 kb fragment of the goat uromodulin promoter and signal sequence

A site for the restriction endonuclease FseI;

Sequences encoding a spider silk protein;

A site for the restriction endonuclease SgfI; and

A 2.8 kb fragment uromodulin 3' genomic DNA.

The pUM/5S13 construct is digested with FseI and SgfI to remove the sequence encoding the spider silk protein. Please refer to PCT Publication No. WO00/15772 (insulator and uromodulin promoter and genomic DNA elements), as well as Lazaris et al. , Science (2002) 295: 472-476 and PCT Publication No. WO99/47661 (spider silk protein constructs), for disclosure of methods to construct pUM/5S13.

PCR is performed on a BChE cDNA clone (ATCC, #65726) with a sense primer (5' CAA TCA GGC CGG CCA GAA GAT GAC ATC ATA ATT GC-3') (SEQ ID NO: 35) containing an FseI site (underlined) and an antisense primer (5' CTA TGA CTC GAG GCG ATC GCT ATT AAT TAG AGA CCC A CAC-3') (SEQ IDNO: 10) including a SgfI site (underlined) to amplify the sequence encoding the mature human BChE protein.

This PCR product is digested with FseI and SgfI, and ligated with the FseI and SgfI fragment of pUM/5S13 to replace the spider silk encoding sequence with the BChE encoding sequence. This new construct is named pUM/BChE.

For microinjection or transfection, XhoI and NotI digestion of pUM/BChE removes the vector backbone and generates a linear DNA fragment. This fragment consists of the insulator, the uromodulin promoter and signal sequence, the BChE-encoding sequence, and a uromodulin 3' genomic DNA fragment.

8.2.Uroplakin II promoter

A group of membrane proteins known as uroplakins are produced on the apical surface of the urothelium. The term "urothelium" refers collectively to the epithelial lining of the ureter, bladder, and urethra. These uroplakin proteins form two-dimensional crystals, known as "urothelial plaques," which cover over 80% of the apical surface of urothelium (Sun et al. , Mol. Biol. Rep. (1996) 23:3-11; Yu et al. , J. Cell Biol. (1994) 125:171-182). These proteins are urothelium-specific markers, and are conserved during mammalian evolution (Wu, et al. J. Biol. Chem. (1994) 269:13716-13724).

Transgenic mice that express human growth hormone (hGH) under the control of the mouse uroplakin II gene promoter have been generated. These mice express the recombinant hGH in the urothelium, and secrete the recombinant hGH into their urine at a concentration of 100-500 mg/l (Kerr et al. , Nat. Biotechnol. (1998) 16:75-79). This study is apparently the first report of using urothelium as a bioreactor for the production and secretion of bio-active molecules. It has subsequently been shown that urothelial cells are involved in urinary protein secretion (Deng et al. , Proc. Natl. Acad. Sci. USA (2001) 98:154-159).

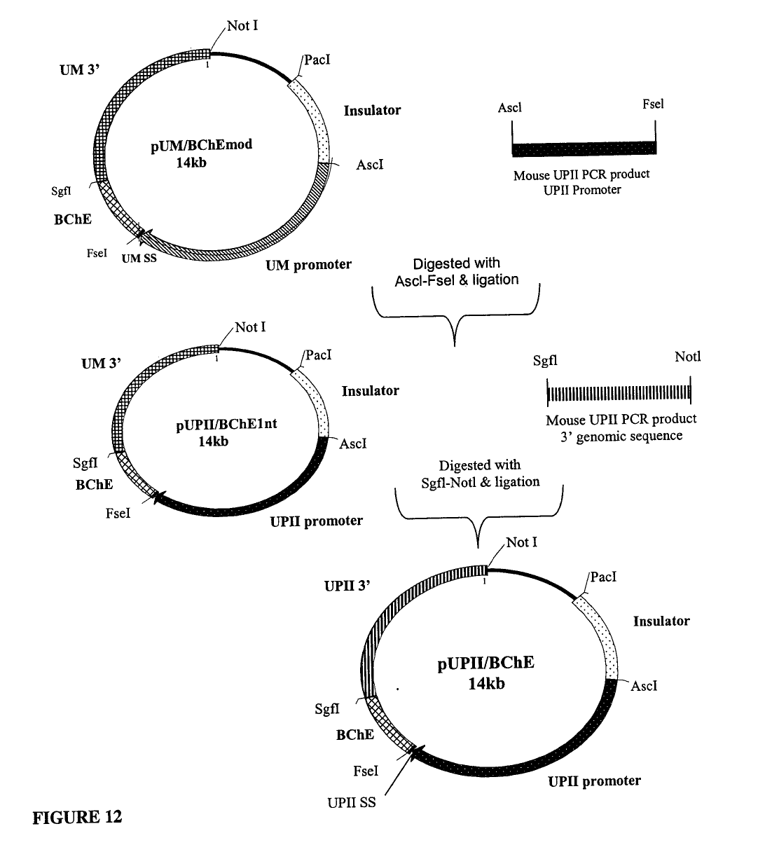

The expression construct pUM/BChE, comprising the uromodulin promoter and sequences encoding a BChE enzyme (See Example 8.1.), may be modified for the construction of the new expression construct pUPII/BChE (See FIGURE 12). The pUM/BChE expression construct contains, in this order: an 2.4 kb fragment of the chicken β-globin insulator; a 3.4 kb fragment of the goat uromodulin promoter and signal sequence; a site for the restriction endonuclease FseI; a BChE-encoding sequence; a site for the restriction endonuclease SgfI; and a 2.8 kb fragment of uromodulin 3' genomic sequence.

Restriction endonuclease sites are introduced at the 5' end (PacI) and the 3' end (AscI) of the chicken β-globin insulator sequence of pUM/BChE by conventional PCR to yield pUM/BChEmod.

PCR is performed on mouse genomic DNA with a sense primer (5' CAA TCA GGC GCG CCC TCG AGG ATC TCG GCC CTC TTT CTG 3') (SEQ ID NO: 36) containing an AscI site (underlined) and an antisense primer (5' CAA TCA GGC CGG CCG CAA TAG AGA CCT GCA GTC CCC GGA G 3') (SEQ ID NO: 37) including a FseI site (underlined) and partial sequence for the signal peptide of the uroplakin II protein. This PCR amplifies a DNA fragment containing the uroplakin II promoter plus the uroplakin signal sequence.

The uroplakin II PCR product is digested with AscI and FseI, and ligated with AscI and FseI digested pUMBChE to replace the goat uromodulin promoter with the mouse uroplakin II promoter. This step generates the construct pUPII/BChEInt.

A PCR is performed on mouse genomic DNA with a sense primer (5' CAT CTG GCG ATC GCT ACC GAG TAC AGA AGG GGA CG-3') (SEQ ID NO: 38) containing a SgfI site (underlined) and an antisense primer (5' CTA GCA TGC GGC CGC GTG CTC TAG GAC AGC CAG AGC-3') (SEQ ID NO: 39) containing a NotI site (underlined) to amplify a portion of the uroplakin II genomic sequence. This PCR product spans uroplakin II genomic sequence from within exon 4 through the 3' end of the gene, including the polyA sequence. This PCR product is digested with SgfI and NotI, and then ligated to SgfI and NotI digested pUPII/BChEInt. This step replaces the goat uromodulin 3' genomic sequences with mouse UPII 3' genomic sequences to generate the final expression construct pUPII/BChE.

For microinjection or transfection, pUPII/BChE is linearized by PacI and NotI to remove the vector backbone. This linear fragment consists of the insulator, the uroplakin II promoter and signal sequence, a BChE-encoding sequence, and a uroplakin II 3' genomic fragment.

CITED REFERENCES

Allon et al. , Toxicol. Sci. (1998) 43:121-128.

Altamirano et al , . J Neurochemistry (2000) 74:869-877.

Andres et al. , . Proc. Natl. Acad. Sci. USA (1987) 84(5):1299-1303.

Arkin et al. , Proc. Natl. Acad. Sci. USA (1992) 89:7811-7815

Ashani et al , . Biochem. Pharmacol. (1991) 41:37-41.

Badgett and Kumar. Urologia Internationalis (1998) 61:72-75.

Baranyi et al. , Gene (1996) 174(1):27-34.

Blong et al. , Biochem. J. (1997) 327:747-757.

Bon et al. , J. Biol. Chem. (1997) 272(5):3016-3021.

Brandeis et al., Pharmacol. Biochem. Behav. (1993) 46:889-896.

Broomfield et al., J Pharmacol. Exp. Ther. (1991) 259:633-638.

Cascio et al., Minerva Anestesiol (1998) 54:337.

Chitlaru et al., Biochem. J. (1998) 336:647-658.

Cibelli et al., Science (1998) 280: 1256-8.

Deng et al., Proc. Natl. Acad. Sci. USA (2001) 98:154-159.

Ebert et al. , Biotechnology (1994) 12:699-702.

Ellman et al. , Biochem Pharmacol (1961) 7:88-95.

Greener et al. , Strategies in Mol. Biol. (1995) 7:32.

Gutierrez et al. , Transgenic Research (1996) 5(4):271-279.

Harduin-Lepers et al. , Biochimie (2001) 83:727-737.

Hennet. Cell Mol. Life Sci. (2002) 59:1081-1095

Hennighausen et al. , Nucl. Acids Res. (1982) 10:3733-3744.

Hermes et al. , Proc. Natl. Acad. Sci. USA (1990) 87:696-700.

Hoffman et al. , J Toxicol Clin Toxicol (1996) 34:259-266.

Karnovsky and Roots, Histochem. Cytochem. (1964) 12:219-221.

Keefer et al. , Biol. Reprod. (2001) 64:849-856.

Kerr et al. , Nat. Biotechnol. (1998) 16:75-79.

Krejci et al. , J. Biol. Chem. (1997) 272:22840-22847.

Lebkowski et al. , Mol Cell Biol (1988) 8(10):3988-3996.

Lee et al. , Nucleic Acids Res. (1988) 16:1027-1041.

Lockridge and La Du, J Biol Chem (1978) 253:361-366.

Lockridge et al. , Biochemistry (1997) 36:786-795.

Lockridge et al. , Biochemistry (1997) 36(4):786-795.

Lynch et al Toxicol Appl Pharmacol (1997) 145:363-371.

Masson et al. , J Biol Chem (1993) 268(19):14329-41.

McClenaghan et al. , Biochem J (1995) 310(Pt2):637-641.

McKnight et al. , Proc Natl Acad Sci USA (1992) 89:6943-6947.

McKnight et al. , Mol Reprod Dev (1996) 44(2):179-184.

McTiernan et al. , Proc. Natl. Acad. Sci USA (1987) 84:6682-6686.

Oliphant et al. , Gene (1986) 44:177-183.

Page et al. , Transgenic Res (1995) 4(6):353-360.

Platteborze and Broomfield. Biotechnol Appl. Biochem (2000) 31:225-229.

Prody et al. , Proc. Natl. Acad. Sci. USA (1987) 84:3555-3559.

Raveh et al. , Biochem Pharmacol (1993) 45(12):2465-2474.

Raveh et al. , Toxicol. Appl. Pharmacol. (1997) 145:43-53.

Rosenberg et al. , Life Sciences (2002) 72:125-134.

Saxena et al. , Biochemistry (1997) 36:7481-7489.

Saxena et al. , Molec. Pharmacol. (1998) 53:112-122.

Sikri et al. , Biochem. J. (1985) 225:481-486.

Soreq et al. , EMBO Journal 1984 3(6):1371-1375.

Soreq et al. , J. Biol. Chem. (1989) 264:10608-10613.

Stacey et al. , Mol. Carcinog. (1990) 3:216-225.

Sun et al. , Mol. Biol. Rep. (1996) 23:3-11.

Takashima et al. , J. Biol. Chem. (2002) 277:45719-45728.

Velander et al., Proc. Natl. Acad. Sci. USA (1992) 89:12003-12007.

Vilotte et al., Eur. J. Biochem. (1989) 186: 43-48.

Wang et al., Molecular Reproduction and Development (2002) 63:437-443.

Wei et al., Biochem Pharmacol (2000) 60(1):121-126.

Wilmut et al., Nature (1997) 385: 810-813.

Wolfe et al., Toxicol Appl Pharmacol (1992) 117(2):189-193.

Wu et al., J. Biol. Chem. (1994) 269:13716-13724.

Xie et al., Molec. Pharmacol. (1999) 55:83-91.

Yu et al., J. Cell Biol. (1994) 125:171-182.

Yuan et al., Acta Pharmacologica Sinica, (1999) 20:74-80.

Zbikowska et al., Biochem J (2002) 365(Pt1):7-11.

Zbikowska et al., Transgenic Res 2002 11(4):425-435.

***

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for in the description.

Patents, patent applications, publications, product descriptions, and protocols are cited throughout this application, the disclosures of which are incorporated herein by reference in their entireties for all purposes.

What is Claimed is:

1. A non-human transgenic mammal that upon lactation, expresses a BChE enzyme in its milk, wherein the genome of the mammal comprises a DNA sequence encoding a BChE enzyme, operably linked to a mammary gland-specific promoter, and a signal sequence that provides secretion of the BChE enzyme into the milk of the mammal.

2. The transgenic mammal of claim 1, wherein the genome of the mammal further comprises a DNA sequence encoding a glycosyltransferase, operably linked to a mammary gland-specific promoter, and a signal sequence that provides secretion of the glycosyltransferase.

3. The transgenic mammal of claim 1 wherein the mammary gland-specific promoter is a casein promoter or a whey acidic protein (WAP) promoter.

4. A non-human transgenic mammal that expresses a BChE enzyme in its urine, wherein the genome of the mammal comprises a DNA sequence encoding a BChE enzyme, operably linked to a urinary endothelium-specific promoter, and a signal sequence that provides secretion of the BChE enzyme into the urine of the mammal.

5. The transgenic mammal of claim 4, wherein the genome of the mammal further comprises a DNA sequence encoding a glycosyltransferase, operably linked to a urinary endothelium-specific promoter, and a signal sequence that provides secretion of the glycosyltransferase.

6. The transgenic mammal of claim 4, wherein the urinary endothelium-specific promoter is a uroplakin promoter or a uromodulin promoter.

7. The transgenic mammal of claim 1 or 4, wherein the mammal is a goat or a rodent.

8. The transgenic mammal of claim 7, wherein the mammal is a goat.

9. The transgenic mammal of claim 1 or 4, wherein the BChE enzyme is a human BChE.

10. The transgenic mammal of claim 9, wherein the human BChE has an amino acid sequence as depicted in SEQ ID NO: 1.

11. The transgenic mammal of claim 1 or 4, wherein the BChE enzyme is a fusion protein.

12. The transgenic mammal of claim 11, wherein the BChE enzyme is fused to human serum albumin.

13. A genetically-engineered DNA sequence, which comprises: (i) a sequence encoding a BChE enzyme; (ii) a mammary gland-specific promoter that directs expression of the BChE enzyme; and (iii) at least one signal sequence that provides secretion of the expressed BChE enzyme.

14. The genetically-engineered DNA sequence of claim 13, wherein the promoter is a mammary gland-specific promoter selected from the group consisting of a WAP (whey acidic protein) promoter and a casein promoter.

15. A method for making a genetically-engineered DNA sequence, which method comprises joining a sequence encoding a BChE enzyme with a mammary gland-specific promoter the directs expression of the BChE enzyme and at least one signal sequence that provides secretion of the expressed BChE enzyme.

16. A genetically-engineered DNA sequence, which comprises: (i) a sequence encoding a BChE enzyme; (ii) a urinary endothelium-specific promoter that directs expression of the BChE enzyme; and (iii) at least one signal sequence that provides secretion of the expressed BChE enzyme.

17. The genetically-engineered DNA sequence of claim 16, where the promoter is a urinary endothelium-specific promoter selected from the group consisting of a uroplakin promoter or a uromodulin promoter.

18. A method for making a genetically-engineered DNA sequence, which method comprises joining a sequence encoding a BChE enzyme with a urinary endothelium-specific promoter that directs expression of the BChE enzyme and at least one signal sequence that provides secretion of the expressed BChE enzyme.

19. The genetically-engineered DNA sequence of claim 13 or 16, wherein the encoded human BChE has an amino acid sequence as depicted in SEQ ID NO: 1.

20. The genetically-engineered DNA sequence of claim 13 or 16, wherein the sequence encoding the BChE has a nucleic acid sequence as depicted in SEQ ID NO: 2.

21. A mammalian cell which comprises the DNA sequence of claim 13.

22. The mammalian cell of claim 21, wherein the cell is a MAC-T (mammary epithelial) cell.

23. A mammalian cell which comprises the DNA sequence of claim 16.

24. The mammalian cell of claim 23, wherein the cell is a BHK (baby hamster kidney) cell.

25. The mammalian cell of claim 21 or 23, wherein the cell is selected from the group of embryonic stem cells, embryonal carcinoma cells, primordial germ cells, oocytes, or sperm.

26. A non-human mammalian embryo which comprises the DNA sequence of claim 13.

27. A non-human mammalian embryo which comprises the DNA sequence of claim 16.

28. A method for producing a transgenic mammal that upon lactation secretes a BChE enzyme in its milk, which method comprises allowing an embryo, into which at least one genetically-engineered DNA sequence, comprising (i) a sequence encoding a BChE enzyme; (ii) a mammary gland-specific promoter; and (iii) a signal sequence that provides secretion of the BChE enzyme into the milk of the mammal, has been introduced, to grow when transferred into a recipient female mammal, resulting in the recipient female mammal giving birth to the transgenic mammal.

29. The method of claim 28, which further comprises introducing the genetically-engineered DNA sequence into a cell of the embryo, or into a cell that will form at least part of the embryo.

30. The method of claim 29, wherein introducing the genetically-engineered DNA sequence comprises pronuclear or cytoplasmic microinjection of the DNA sequence.

31. The method of claim 29, wherein introducing the genetically-engineered DNA sequence comprises combining a mammalian cell stably transfected with the DNA sequence with a non-transgenic mammalian embryo.

32. The method of claim 29, wherein introducing the genetically-engineered DNA sequence comprises the steps of
(a) introducing the DNA sequence into a non-human mammalian oocyte; and
(b) activating the oocyte to develop into an embryo.

33. A method for producing a transgenic mammal that upon lactation secretes a BChE enzyme in its milk, which method comprises cloning or breeding of a transgenic mammal, the genome of which comprises a DNA sequence encoding a BChE enzyme, operably linked to a mammary gland-specific promoter, wherein the sequence further comprises a signal sequence that provides secretion of the BChE enzyme into the milk of the mammal.

34. A method for producing a transgenic mammal that secretes a BChE enzyme in its urine, which method comprises allowing an embryo, into which at least one genetically-engineered DNA sequence, comprising (i) a sequence encoding a BChE enzyme; (ii) a urinary endothelium-specific promoter; and (iii) a signal sequence that provides secretion of the BChE enzyme into the urine of the mammal, has been introduced, to grow when transferred into a recipient female mammal, resulting in the recipient female mammal giving birth to the transgenic mammal.

35. The method of claim 34, which further comprises introducing the genetically-engineered DNA sequence into a cell of the embryo, or into a cell that will form at least part of the embryo.

36. The method of claim 35, wherein introducing the genetically-engineered DNA sequence comprises pronuclear or cytoplasmic microinjection of the DNA sequence.

37. The method of claim 35, wherein introducing the genetically-engineered DNA sequence comprises combining a mammalian cell stably transfected with the the DNA sequence with a non-transgenic mammalian embryo.

38. The method of claim 35, wherein introducing the genetically-engineered DNA sequence comprises the steps of
(a) introducing the DNA sequence into a non-human mammalian oocyte; and
(b) activating the oocyte to develop into an embryo.

39. A method for producing a transgenic mammal that secretes a BChE enzyme in its urine, which method comprises cloning or breeding of a transgenic mammal, the genome of which comprises a DNA sequence encoding a BChE enzyme, operably linked to a urinary endothelium-specific promoter, wherein the sequence further comprises a signal sequence that provides secretion of the BChE enzyme into the urine of the mammal.

40. A method for producing a BChE enzyme, which method comprises:
(a) inducing or maintaining lactation of a transgenic mammal, the genome of which comprises a DNA sequence encoding a BChE enzyme, operably linked to a mammary gland-specific promoter, wherein the sequence further comprises a signal sequence that provides secretion of the BChE enzyme into the milk of the mammal; and
(b) extracting milk from the lactating mammal.

41. The method according to claim 40, which comprises the additional step of isolating the BChE enzyme from the extracted milk.

42. The method according to claim 41, further comprising purifying the BChE enzyme.

43. The milk of a non-human mammal comprising a human BChE enzyme.

44. Milk comprising a BChE enzyme produced by a transgenic mammal according to the method of claim 40.

45. The milk of claim 43 or 44, where the milk is whole milk.

46. The milk of claim 43 or 44, where the milk is defatted milk.

47. A method for producing a BChE enzyme, which method comprises extracting urine from a transgenic mammal, the genome of which comprises a DNA sequence encoding a BChE enzyme, operably linked to a urinary endothelium-specific promoter, where the sequence further comprises a signal sequence that provides secretion of the BChE enzyme into the urine of the mammal.

48. The method according to claim 47, which comprises the additional step of isolating the BChE enzyme from the extracted urine.

49. The method according to claim 48, further comprising purifying the BChE enzyme.

50. Urine of a non-human mammal comprising a human BChE enzyme.

51. Urine comprising a BChE enzyme produced by a transgenic mammal according to the method of claim 47.

52. A method for producing a BChE enzyme in a culture of MAC-T or BHK cells, which method comprises:
(a) culturing said cells, into which a DNA sequence comprising (i) a DNA sequence encoding a BChE enzyme, (ii) a promoter that provides expression of the encoded BChE enzyme within said cells, and (iii) a signal sequence that provides secretion of the BChE enzyme into the cell culture medium, has been introduced;
(b) culturing the cells; and
(c) collecting the cell culture medium of the cell culture.

53. The method of claim 52, which comprises the additional step of isolating the BChE enzyme from the collected cell culture medium.

54. The method according to claim 53, further comprising purifying the BChE enzyme.

55. The method of claim 52, wherein the cells are MAC-T cells and at least 50% of the produced BChE enzyme is in tetramer form.

56. Cell culture medium comprising a BChE enzyme produced by cultured MAC-T or BHK-1 cells according to the method of claim 52.

57. Cell culture medium from a culture of mammalian cells, which medium comprises a BChE enzyme, wherein at least 50% of the BChE enzyme is in tetramer form.

58. A method for producing a pharmaceutical composition, which comprises combining
(a) a BChE enzyme produced by a transgenic mammal according to the method of claim 40, 41, 42, 47, 48, or 49 with
(b) a pharmaceutically acceptable carrier or excipient.

59. A method for producing a pharmaceutical composition, which comprises combining
(a) a BChE enzyme produced in a culture of MAC-T or BHK cells according to the method of claim 52 with
(b) a pharmaceutically acceptable carrier or excipient.

60. A method for the treatment of organophosphate poisoning, which comprises administering to a subject in need thereof a therapeutically effective amount of a pharmaceutical composition produced by the method of claim 58 or 59.

61. A method for the treatment of post-surgical, succinyl choline-induced apnea, which comprises administering to a subject in need thereof a therapeutically effective amount of a pharmaceutical composition produced by the method of claim 58 or 59.

62. A method for the treatment of cocaine intoxication, which comprises administering to a subject in need thereof a therapeutically effective amount of a pharmaceutical composition produced by the method of claim 58 or 59.
